(19) United States
(12) Patent Application Publication
(10) Pub. No.: US 2014/0193456 A1
(43) Pub. Date: Jul. 10, 2014

(54) Method for Drying-Conservation of Natural Substances

(71) Applicant: Hristem Mitkov Dyanov, Niles, IL (US)

(72) Inventors: Hristem Mitkov Dyanov, Niles, IL (US); Paoula Hristemova Dyanova, Skokie, IL (US)

(21) Appl. No.: 14/150,648

(22) Filed: Jan. 8, 2014

Related U.S. Application Data
(60) Provisional application No. 61/750,518, filed on Jan. 9, 2013.

(51) Int. Cl. A01N 3/00; A61K 36/09; A61K 36/06; A61K 36/00; A61K 36/02

(52) U.S. Cl. CPC A01N 3/00; A61K 36/00; A61K 36/02; A61K 36/06; A61K 36/09
USPC 424/195.15; 424/725; 435/420; 424/195.17; 800/298

(57) ABSTRACT

The invention comprises of an implementation of the lyophilization process in a method for a large-scale preservation of the entire biological material prior to the extraction and/or the utilization/manipulation of any substances of the biological content. The drying-conservation of all the natural substances is achieved via deep-freezing evaporation followed by complete drying of the biological material by the means of sublimation, and other additional manipulations and procedures suitable for the preparation of various commercially viable products further on.

### CROSS-REFERENCE TO RELATED APPLICATIONS

This application is claiming the priority benefit of the provisional patent application U.S.-61/750,518 “Method for Drying-Conservation of Natural Substances”.

### DESCRIPTION

### Terminology And Abbreviations Used

Cell Packing Effect: The effect on the survival of cells when frozen at a high, rather than a low, packing density.
Chilling Injury: Injury that occurs as a result of a reduction in temperature.
Colligative Effect: A physical property of a system that depends on the number of molecules and not their nature.
Cryopreservation: The storage of a living organism, or a portion thereof, at an ultralow temperature (typically colder than −130° C.) such that it remains capable of survival upon thawing.
Cryoinjury: Damage caused by reduction in temperature irrespective of the mechanism.
Cryoprotectant or Cryoprotective Agent (CPA): A substance that protects a living system against injury due to reduction in temperature.
Cryostorage: The storage of a living organism, or a portion thereof, at an ultra-low temperature (typically colder than −130° C.) such that it remains capable of survival upon thawing.
Eutectic Temperature: The lowest temperature (for a crystallizing solute) at which the existence of a liquid phase for a given system is possible.
Freezing: The crystallization of liquid water to form ice.
Freeze-Drying: A controllable method of dehydrating labile products by vacuum desiccation (also termed as lyophilization).
Glass Transition Temperature (Tg): The temperature (for an amorphous solute) where the residual liquid vitrifies in the presence of ice.
Intracellular Freezing: The formation of ice crystals within cells.
Lyophilization: A controllable method of dehydrating labile products by vacuum desiccation (also termed “freeze-drying”).
Melting Point: The temperature during the warming of an aqueous system at which the last ice melts. This temperature is equal to the equilibrium freezing point.
Nucleation: The formation of a nucleus upon which an ice crystal can grow; this may be an appropriate arrangement of water molecules or a foreign particle.
Solution Effects: Damage to cells that is a result of the increase in solute concentration that occurs as a secondary effect of freezing.
Super-Cooling: Reduction of temperature below the equilibrium freezing point but without freezing, hence, an unstable situation. Note: supercooling is often not hyphenated.
Vitrification: The conversion of an aqueous system to an amorphous, noncrystalline solid solely by increase in viscosity.

### FIELD OF THE INVENTION

This invention relates to methods of preserving, shredding, sampling, encapsulating, extracting and purifying bioactive substances from various biological origins. More specifically, it aligns to methods of preserving, extracting, manipulating and separating bioactive substances from various biological sources using deep freezing, fluid extraction and/or hydrocarbon solvent extract. The invention further relates to methods for manipulation and separation of bioactive substances contained in biological materials and extracts using instant freezing by direct contact to the biological material. The presented invention also relates to formulations, pharmaceutical preparations and dietary supplements that may be prepared with the original biological material and/or extracted bioactive substances and a use of such pharmaceutical preparations and dietary supplements to treat various human ailments and for cosmetic purposes. Most importantly, the invented process of biological material preservation and conservation is intended for commercial-scale manipulations and market value product preparations with no particular implementation for laboratory- and investigation practices and purposes alone.

### BACKGROUND OF THE INVENTION

Throughout history, humans have ingested and otherwise consumed a wide variety of plants, herbs, and extracts of such plants and herbs to help alleviate aches and pains, improve immunity to infection, treat various illnesses, or even to induce relaxation or stress reduction.

Although many types of herbs and fungi have been identified and used through the human civilization history for medicinal and cosmetic purposes, no simple and efficient method is available for both efficient preservation of all bioactive components within the biological material and for extraction of bio-components from the roots and other parts of the material, and separation of each individually extracted component. Traditionally, herbs are air-dried at an ambient temperature of choice and different extraction methods have been employed including boiling and steam distillation to obtain components suspected to have biologically important properties. In many cases plant slurry has been steam distilled and the first portion of distillate has been collected, filtered and lyophilized. Alternately, a liquid-solid extraction at room temperature has been reported wherein the above slurry has been intimately mixed in a blender and the mixture then has been filtered and lyophilized. In many recent cases, rather than lyophilization, the filtrate has been subjected to successive extractions with organic solvents such as chloroform and else. These purification operations removed impurities from the aqueous layer. The extraction yield for these methods varied depending on the solvent and methodology used. Importantly, most of the approaches employed for manipulating the biological material are known to have harmful effect on most of the components of the material leading to total loss or particular degradation of their biological activities resulting in quite limited pharmaceutical gains.

Much more practical approach is proposed in the methodology described in this invention for nearly-perfect preservation of the entire biological content obtained and for preparation of commercially viable products—preserving for future utilization all the biologically active components as good as they exist in vivo (alive) where particular separation of certain bio-chemicals is not especially desired. We employ rapid and instant freezing of the biological objects under conditions leading to residual water “glassification” (or vitrification—instant freezing without crystallization and volume increase) followed by water evaporation, other manipulations and encapsulation of the biological material as a commercial product without any treatments harmful for degrading the bio-chemical content nor cellular structure leading to supplements as natural as the alive original obtained initially.

### The Origins of Freeze Drying

The method can be traced back to prehistoric times and has been used by the Aztecs and Eskimo for preserving foodstuffs. The Andean civilizations had preserved potatoes using a freeze-drying process. They called this foodstuff Chuño. Toward the end of the 1880s the process has been used on animal material on a laboratory scale and the basic principles—well understood at that time. Practically, the method remained a laboratory technique until the 1930s when there was the need to process heat-labile antibiotics and blood products. At this time, refrigeration and vacuum technologies had advanced sufficiently to enable production freeze-dryers to be developed, and since then the process has been used industrially in both the food and pharmaceutical industries. Freeze-drying was actively developed during WWII. Serum being sent to Europe for medical treatment of the wounded required refrigeration, but because of the lack of simultaneous refrigeration and transport, many serum supplies were spoiling before reaching their intended recipients. Therefore, the freeze-drying process was developed as a commercial technique that enabled serum to be rendered chemically stable and viable without having to be refrigerated. Shortly thereafter, the freeze-dry process was applied to penicillin and bone, and lyophilization became recognized as an important technique for preservation of biologicals. Since that time, freeze-drying has been used as a preservation or processing technique for a wide variety of products. These applications include the following but are not limited to the processing of food, pharmaceuticals and diagnostic kits, and animal and human material; the restoration of water damaged documents; the preparation of river-bottom sludge for hydrocarbon analysis; the manufacturing of ceramics used in the semiconductor industry; the production of synthetic skin; the restoration of historic/reclaimed boat hulls.

### Vitrification

The conversion of an aqueous system to an amorphous, noncrystalline solid solely by increase in viscosity is called “Vitrification”. The vitrification is defined by the viscosity of the solution reaching a sufficiently high value (˜1013 poises) to behave like a solid but without crystallization. In conventional cryopreservation, the concentration of solute in the remaining liquid increases during progressive freezing, and a temperature (Tg) is eventually reached with many systems where the residual liquid vitrifies in the presence of ice (FIG. 1, black arrow). Cells can survive this situation, they do so in conventional cryopreservation, but they will not tolerate exposure to the necessary concentration for vitrification without freezing (˜80 g % [w/w]) at temperatures above 0° C. Some other solutes will vitrify at lower concentrations, for example butane-2,3-diol at around 35% (w/w), but unfortunately this compound is more toxic than glycerol. Luyet [1] knew that it was possible to vitrify solutions that are less concentrated than this if sufficiently rapid cooling was employed; the reason is that ice crystals form by the accretion of water molecules onto a nucleus (i.e. a center of crystal formation process). Both the formation of the nuclei and the subsequent growth of ice crystals are temperature dependent. Nucleation is unlikely just below the equilibrium freezing point (hence the phenomenon of super-cooling), but it becomes more probable as the temperature falls, reaches a maximum rate, and then decreases as the movement of water is limited by viscosity. However, the growth of ice crystals is maximal just below the freezing point and is progressively slowed, and eventually arrested, by cooling. The interaction of these two processes creates three possibilities for a cooled sample (FIG. 2); if it cools rapidly it may escape both nucleation and freezing; if it cools sufficiently slowly it will nucleate and then freeze; and at an intermediate cooling rate it will nucleate but not freeze. Upon warming, however, there are only two possibilities; if heated sufficiently rapidly it will escape both nucleation and freezing during warming; the alternative is that the trajectory passes through both the nucleation and the ice crystal growth zones and, therefore, it will nucleate (if it is not already nucleated) and the ice crystals will then grow before eventually melting. Therefore, unless a sufficient concentration of cryoprotectant has been used to ensure that no ice can form under any circumstances, there is a risk that freezing will occur during warming. The problem is that bulky tissues and organs cannot be cooled much more rapidly than a few degrees per minute in practice. The demonstration that ice forming in tissues produces so much damage has created renewed interest in the possibility of using vitrification with very high concentrations of appropriate cryoprotectants to avoid the formation of ice completely. Current research aims to identify materials that will inhibit the formation of ice crystals during warming [2,3], and one interesting possibility is the antifreeze proteins that some polar fish and overwintering insects have evolved to avoid freezing in nature. One effect of such compounds is to reduce the warming rate required to prevent ice crystallization to more manageable rates. This approach is being used in conjunction with electromagnetic heating [4,5] to achieve more rapid and more uniform heating. However, despite progress in the design of vitrification cocktails with reduced toxicity, the major problem remains cryoprotectant toxicity. One approach to this problem is to increase the concentration of cryoprotectant progressively during cooling so that the tissue concentration follows the liquids curve: ice does not form but the cells do not experience any greater concentration of cryoprotectant than occurs during freezing. This has recently proved to be practical and very effective for the cryopreservation of articular cartilage, an otherwise recalcitrant tissue [6]. The same method may potentially be effective for other resistant tissues and perhaps even for organs.

### Defining Freeze-Drying

Freeze-drying or lyophilization describe precisely the same process. The term “lyophilization,” which means “to make solvent loving,” is less descriptive than the alternative definition “freeze-drying.” Several alternative definitions have been used to describe freeze-drying. Operationally we could define freeze-drying as a controllable method of dehydrating labile products by vacuum desiccation.

Earlier accounts of freeze-drying suggested that ice was only removed by sublimation and defined this step as primary drying. The cycle was then described as being extended by secondary drying or desorption. Although these definitions are applicable to ideal systems, they incompletely define the process for typical systems that form an amorphous matrix or glass when cooled.

Technically, freeze-drying may be defined as:
1. Cooling of the liquid sample followed by the conversion of freezable solution water into ice, crystallization of crystallizable solutes, and the formation of an amorphous matrix comprising non-crystallizing solutes associated with unfrozen moisture.
2. Sublimation of ice under vacuum.
3. “Evaporation” of water from the amorphous matrix.
4. Desorption of chemi-absorbed moisture resident in the apparently dried cake.

Freeze-drying has a number of advantages over alternative stabilizing methods. These may be summarized by the following criteria:
1. The need to stabilize materials for storage or distribution.
2. The product may demand to be freeze-dried and there may be no suitable alternative available.
3. There may be a legal requirement to freeze-dry the product to satisfy regulatory demands.
4. Freezing will reduce thermal inactivation of the product and immobilize solution components.
5. Concentration effects such as “salting out” of proteins, alterations in the distribution of components within the drying and dried product, and so on, may be minimized by freeze-drying.
6. The water content of the dried product can be reduced to low levels, and in general samples are more shelf-stable when dried to low moisture contents, although over-drying may reduce shelf stability in sensitive biomaterials.
7. Because the product is normally sealed under vacuum or an inert gas, oxidative denaturation is reduced.
8. Loss of water equates to a loss of product weight and this may be important where transport costs are significant.
9. Sample solubility, shrinkage, unacceptable appearance, or loss of activity may all be improved when freeze-drying is used rather than an alternative technique.
10. Dispensing accuracy may be facilitated when the sample is dispensed as a liquid rather than a powder.
11. Particulate contamination is often reduced when samples are freeze-dried rather than spray or air-dried.
12. The need to compete with competitors supplying similar products.
13. The requirement to launch a product on the market while less costly drying techniques are being developed.
14. The production of intermediate bulk or requirement to remove solvents such as ethanol.
15. The need to maximize investment in drying plants by freeze-drying a minor product rather than invest in an alternative and costly drying process.
16. The need to separately dry two or more components that would be incompatible if dispensed together within a single container.

### Types of Freeze-Dried Products

Freeze-dried products may be classified as:
1. Non-biologicals, where the process is used to dehydrate or concentrate reactive or heat-sensitive chemicals.
2. Nonliving bio-products. These comprise the major area of application and include enzymes, hormones, antibiotics, vitamins, blood products, antibiotics, inactivated or attenuated vaccines, and so on. This subgroup includes pharmaceuticals, which may be used diagnostically or therapeutically.
3. Bone and other body tissues for surgical or medical use; foods where organoleptic properties are important; industrial bio-products.
4. Living organisms for vaccine or seed culture use, which must grow and multiply to produce new progeny after drying and reconstitution, or for herbal, nutritional and/or supplemental utilization.
5. Miscellaneous, for example flood-damaged books, museum artifacts, and so on.

However, freeze-drying is less appropriate for:
1. Oily or sugar-rich materials where the medium does not freeze.
2. Products that form impervious surface skins, thereby preventing vapor migration from the drying sample during processing.
3. Eukaryote cells, which are able to retain viability when frozen only in the presence of additives, may be incompatible with the freeze-drying process.

### Processing Principles of Freeze-Drying

Freeze-drying is a complex process during which drying may proceed more or less rapidly within individual samples throughout the process batch, such that parts of the product will be frozen, whereas other areas are drying or will have dried depending on the nature of the sample and stage in the cycle. The precise freezing and drying behavior will be determined by the interrelationship between the sample and shelf temperature, system pressure, extent of product dryness, and variations in drying conditions throughout the cycle.

Often regarded as a gentle method of drying materials, freeze-drying is in reality a potentially damaging process where the individual process stages should be regarded as a series of interrelated stresses, each of which can damage sensitive bio-products. Damage sustained during one step in the process may be exacerbated at succeeding stages in the process chain and even apparently trivial changes in the process, such as a change in container, may be sufficient to transform a successful process to one which is unacceptable.

Freeze-drying will not reverse damage incurred prior to formulation and care must be exercised when selecting an appropriate cell type or technique used to culture or purify the cell or its extracts prior to freeze-drying. The essence of the formulation exercise should be to minimize freeze-drying damage, loss of viability, or activity. To ensure minimal losses of activity, the sample may require dilution in a medium containing protective additives, specifically selected for the product or application. Although frequently described as “protectants” these additives may not be effective at all stages of the process but may protect only during particular steps in the drying cycle. At other stages, the additive may fail to protect the active component and indeed may be incompatible with the process. It is also important to appreciate that individual stages in the process can result in damage, which initially remains undetected, becoming evident only when the dried sample is rehydrated. Particular attention must be applied to the selection and blending of the additive mixes in the formulation and the importance of formulation will be discussed at greater length later.

Freeze-dried products should be:
1. Minimally changed by the process.
2. Dry.
3. Active.
4. Shelf stable.
5. Clean and sterile (for pharmaceutical applications).
6. Ethically acceptable.
7. Pharmaceutically elegant.
8. Readily soluble and simple to reconstitute.
9. Process should be economically practicable.

Products should be formulated to ensure batch product uniformity, whereas there may be particular requirements relating to product use. In this context, vaccines freeze-dried for oral or aerosol delivery may require the inclusion of excipients that minimize damage when the dried product is exposed to moist air.

A wide range of containers can be used to freeze-dry vaccines, microorganisms, and others, including all glass ampoules, rubber stoppered vials, double chambered vials, and prefilled syringes that hold both dried vaccine and diluent, bifurcated needles, and so on. Alternatively, vaccines can be dried in bulk in stainless steel or plastic trays and the resultant powder tableted, capsulated, sachet filled, or dispensed into aerosol devices for lung or nasal delivery.

### Processing Principles

Freeze-drying is a complex process during which drying may proceed more or less rapidly within individual samples throughout the process batch, such that parts of the product will be frozen, whereas other areas are drying or will have dried depending on the nature of the sample and stage in the cycle. The precise freezing and drying behavior will be determined by the interrelationship between the sample and shelf temperature, system pressure, extent of product dryness, and variations in drying conditions throughout the cycle.

Often regarded as a gentle method of drying materials, freeze-drying is in reality a potentially damaging process where the individual process stages should be regarded as a series of interrelated stresses, each of which can damage sensitive bio-products. Damage sustained during one-step in the process may be exacerbated at succeeding stages in the process chain and even apparently, trivial changes in the process, such as a change in container, may be sufficient to transform a successful process to one that is unacceptable.

Freeze-drying will not reverse damage incurred prior to formulation and care must be exercised when selecting an appropriate cell type or technique used to culture or purify the cell or its extracts prior to freeze-drying. The essence of the formulation exercise should be to minimize freeze-drying damage, loss of viability, or activity. To ensure minimal losses of activity, the sample may require dilution in a medium containing protective additives, specifically selected for the product or application. Although frequently described as “protectants” these additives may not be effective at all stages of the process but may protect only during particular steps in the drying cycle. At other stages, the additive may fail to protect the active component and indeed may be incompatible with the process. It is also important to appreciate that individual stages in the process can result in damage, which initially remains undetected, becoming evident only when the dried sample is rehydrated. Particular attention must be applied to the selection and blending of the additive mixes in the formulation, and the importance of formulation will be discussed at greater length later.

Freezing refers to the abrupt phase change when water freezes as ice. Except for very complex biomolecules or cold sensitive cells, cooling in the absence of freezing (chilling) is generally not damaging to biomaterials.

When solutions or suspensions are frozen, they may cool appreciably below their measured freezing point prior to ice formation, a phenomenon defined as super-cooling (undercooling or sub-cooling). The extent of super-cooling depends on cooling rate, sample composition and cleanliness, dispensed fill volume, container type, method of sample cooling, and so on. Even when a simple solution is repeatedly cooled or warmed, the onset and extent of super-cooling will vary from cycle to cycle. In the super-cooled state, while the composition of the solution remains unchanged, the cooled liquid is thermodynamically unstable and sensitive to ice formation. As the solution is cooled to lower temperatures, the probability of ice crystallization will correspondingly increase. For optimized freeze-drying, the intention should be to induce super-cooling in the suspension to encourage uniform cooling and freezing throughout the sample contents.

Sample freezing may be defined as the abrupt conversion of the suspension into a mixture of ice and solute concentrate. Freezing is a two-step process during which water initially nucleates, followed by the growth of the ice crystals that pervade the solute phase resulting in a mixture of ice and solute concentrate. Under typical processing conditions, ice nucleates heterogeneously around microscopic particles within the suspension and is encouraged by reducing temperature and agitating the super-cooled suspension to increase the probability of contact between nucleating foci and water clusters. Nucleation depends on the number and physical nature of particulate impurities within the suspension or solution. Ice is a particularly effective nucleation focus and cryobiologists may deliberately seed samples with ice to induce nucleation. Other effective ice nucleators include glass shards and specifically formulated nucleation promoters. Whereas nucleation aids can be added to experimental systems, deliberate attempts to add ice inducers to pharmaceutical materials would be at variance with Good Pharmaceutical Manufacturing Practice.

In contrast to nucleation, ice growth (proliferation) is encouraged by raising the temperature, thereby decreasing the suspension viscosity. Ice nucleation and proliferation are inhibited at temperatures below the glass transition temperature (Tg), whereas above the melting temperature (Tm) the suspension or solution will melt. The consequences and measurements of these parameters are important elements in the formulation exercise.

To facilitate the sublimation of water vapor from the drying mass, the ice crystals should be large, wide, and contiguous, extending from the product base toward its surface, thereby providing an optimized structure for vapor migration. Crystal structures commonly observed during freeze-drying when solutions are frozen in trays or vials include dendritic structuring, where the ice crystal branches continuously from the nucleating focus and the spherulite form, and where sub-branching is discouraged because the solution viscosity is high, or fast rates of cooling are used.

Cooling or freezing rates are defined as slow (suboptimal), rapid (super-optimal), or optimal as assessed by criteria such as post-freezing cell survival or biopolymer activity, and is ambiguous unless conditions are more precisely defined. Cooling rates may be defined in terms of: 1) The rate at which the shelf temperature is cooled per unit time; 2) The rate at which the solution cools per unit time; 3) The depth of liquid within the vial (in mm) which cools per unit time.

The ice and solute crystal structure resulting from sample freeze has a major impact on subsequent freeze-drying behavior, encouraging the sample to dry efficiently or with defects such as melt or collapse depending on freezing rate used. The preferred ice structure comprising large contiguous ice crystals is induced by freezing the sample at a slow rate of 0.2-1.0° C./min. The slow cooling will also induce the crystallization of solutes reluctant to crystallize when faster rates of cooling are used. However, a slow rate of cooling may exacerbate the development of a surface skin, which inhibits sublimation efficiency. Slow cooling can also inactivate a bio-product by prolonging sample exposure to the solute concentrate biomolecules. However, a fast rate of cooling can result in the formation of numerous, small, randomly orientated ice crystals embedded in an amorphous solute matrix, which may be difficult to freeze-dry. Complicating the choice of freezing regimes is the fact that the optimal cooling rate cannot be sustained where the sample fill depth exceeds 10 mm. In short, defining cooling rates often requires a compromise in sample requirements.

### Description of a Common Freeze-Drying Process

The freeze-drying process may be divided into a number of discrete steps that may be summarized as:
1. A suitable medium for freeze-drying may be required for the processing of cell or other bio-products within a variety of preparatory processing steps, e.g., vaccine preparation, extraction, purification, and formulation.
2. Sample freezing, which reduces thermal denaturation of product, immobilizes solution components, and prevents foaming when the vacuum is applied. Freezing also induces a desired ice-crystal structure within the sample, which facilitates drying.
3. Primary drying (sublimation) where conditions must be maintained in the drying chamber to sustain water migration from the sample ice during drying. During primary drying, the sample temperature (strictly freeze-drying interface temperature) must be maintained below the eutectic, glass transition, collapse, or melt temperature as appropriate to minimize sample damage during drying.
4. A secondary drying stage during which resident moisture adsorbed to the apparently dry structure is removed by desorption.
5. Sealing the dried sample in a vacuum or under an inert gas at the end of the process, both of which exclude the entry of reactive, destabilizing, atmospheric gases such as oxygen or carbon dioxide into the dried sample and prevent the ingress of damp air into the freeze-dried sample. (Note that a freeze-dried product will have a vastly expanded dry surface area and is therefore particularly sensitive to air denaturation or moisture re-adsorption.)
6. The samples are then removed from the freeze-dryer, stored and/or distributed for use prior to reconstitution for injection, application, or regrowth.

For convenience, the freeze-drying process may be divided into a number of discrete steps that may be summarized as:
1. For the processing of cell or other bio-products a variety of preparatory processing steps may be required, e.g., vaccine preparation, extraction, purification, and formulation in a suitable medium for freeze-drying.
2. Sample freezing, which reduces thermal denaturation of product, immobilizes solution components, and prevents foaming when the vacuum is applied. Freezing also induces a desired ice-crystal structure within the sample, which facilitates drying.
3. Primary drying (sublimation) where conditions must be maintained in the drying chamber to sustain water migration from the sample ice during drying. During primary drying the sample temperature (strictly freeze-drying interface temperature) must be maintained below the eutectic, glass transition, collapse, or melt temperature as appropriate to minimize sample damage during drying.
4. A secondary drying stage during which resident moisture adsorbed to the apparently dry structure is removed by desorption.
5. Sealing the dried sample in a vacuum or under an inert gas at the end of the process, both of which exclude the entry of reactive, destabilizing, atmospheric gases such as oxygen or carbon dioxide into the dried sample and prevent the ingress of damp air into the freeze-dried sample. (Note that a freeze-dried product will have a vastly expanded dry surface area and is therefore particularly sensitive to air denaturation or moisture re-adsorption.)
6. The samples are then removed from the freeze-dryer, stored and/or distributed for use prior to reconstitution for injection, application, or regrowth.

### The Freeze-Drying Process Practices

The preservation of biological material in a stable state is a fundamental requirement in biological/medical science, agriculture, and biotechnology. It has enabled standardization of experimental work over time, has secured lifesaving banks of cells and tissue ready for transplantation and transfusion at the time of need and has assured the survival of critical germplasm in support of programs for the conservation of species. Cryopreservation and freeze-drying are widely accepted as the preferred techniques for achieving long-term storage, and have been applied to an increasingly diverse range of biological materials. Although the basis for many methodologies is common, many laboratories lack expertise in applying correct preservation and storage procedures and many apply outdated or inappropriate protocols for storing samples or cultures. Importantly, the common lyophilization practices are employing techniques and conditions allowing for water-crystal formation within the biological material for stimulating the evaporation process with reducing the cost, and preventing a direct physical/chemical contact between the biological material and the cooling surfaces for preventing contamination—exactly in opposite to the concept utilized in this invention.

Samples may be frozen in a variety of ways depending on operational requirements:
1. Samples may be frozen in a freezer or a cooling tunnel prior to transfer to the freeze-dryer for desiccation. Advantages include increased annual sample throughput because the freeze-dryer is used only for drying. Disadvantages include the greater risk of sample melt or contamination resulting from the need to transfer samples from the freezer into the drier.
2. Samples may be frozen in the absence of cooling by evacuating the container and relying on evaporative cooling to freeze the sample. However, the need to prevent sample foaming when the dryer is evacuated precludes the widespread use of the method.
3. Pellet freezing. Strictly this is not a method of freezing but can be useful when bulk products, including vaccines for subsequent powder filling, are processed. The suspension is sprayed into a cryogenic liquid or onto a cold surface to form frozen sample droplets, which are then be placed into trays or flasks for freeze-drying. Under these conditions, sublimation rates are typically very high because the thickness of the dry layer is restricted only by the pellet radius, and drying proceeds in a virtually unimpeded manner from each pellet.
4. The most widely used technique is to freeze the samples directly on the freeze-dryer shelf. Although this method has the disadvantage that the drier is used for part of the cycle as freezer, freezing and drying samples within a single machine eliminates the need to transfer samples from freezer to drier, and therefore improves sample cleanliness while reducing product vulnerability.

There are four stages in the complete commercial drying process: pretreatment, freezing, primary drying, and secondary drying.

### Stage 1—Pretreatment

Pretreatment includes any method of treating the product prior to freezing. This may include concentrating the product, formulation revision (i.e., addition of components to increase stability and/or improve processing), decreasing a high vapor pressure solvent or increasing the surface area. In many instances, the decision to pretreat a product is based on theoretical knowledge of freeze-drying and its requirements, or is demanded by cycle time or product quality considerations. Methods of pretreatment include Freeze Concentration, Solution-Phase Concentration, Formulation to Preserve Product Appearance, Formulation to Stabilize Reactive Products, Formulation to Increase the Surface Area, and Decreasing High Vapor Pressure Solvents.^{[1]}

### Stage 2—Freezing

In a lab, freezing is often achieved by placing the material in a freeze-drying flask and rotating the flask in a bath, called a shell freezer, which is cooled by mechanical refrigeration, dry ice and methanol, or liquid nitrogen. On a larger scale, freezing is usually done using a freeze-drying machine. In this step, it is important to cool the material below its triple point, the lowest temperature at which the solid and liquid phases of the material can coexist. This ensures that sublimation rather than melting will occur in the following steps. Larger crystals are easier to freeze-dry. To produce larger crystals, the product should be frozen slowly or can be cycled up and down in temperature. This cycling process is called annealing. However, in the case of food, or objects with formerly-living cells, large ice crystals will break the cell walls (a problem discovered, and addressed, by Clarence Birdseye), resulting in the destruction of more cells, which can result in increasingly poor texture and nutritive content. In this case, the freezing is done rapidly, in order to lower the material to below its eutectic point quickly, thus avoiding the formation of ice crystals. Usually, the freezing temperatures are between −50° C. and −80° C. The freezing phase is the most critical in the whole freeze-drying process, because the product can be spoiled if badly done.

Regarded as the first step in the process, the formulated product must be frozen before evacuating the chamber to induce sublimation. Freezing will:
1. Immobilize the components in the solution and prevent foaming as the vacuum is applied.
2. Reduce thermal inactivation of the dispensed product.
3. Induce a specific ice-crystal structure within the frozen mass, which will facilitate or inhibit vapor migration from the drying cake. In short, the ice structure formed during freezing will dictate subsequent freeze-drying behavior and the ultimate morphology of the dried cake.

Ideally freezing should minimize solute concentration effects and result in a sample where all the components are spatially arranged as in the dispensed solution. However, it may not be possible to achieve this ideal when typical solutions or suspensions are frozen. When addressing the freezing of aqueous solutions or suspensions, there is the need to consider both the solvent (water in the case of aqueous solutions) and solute(s) in the formulation. Frequently, the terms cooling and freezing are erroneously interchanged and confusion in understanding the process may occur and may be compounded by failing to distinguish between shelf or product cooling and freezing. Cooling refers to the reduction of temperature of the freeze-dryer shelves, the diathermy fluid circulating through the shelves, the vial and tray mass, interior of the freeze-dryer, and the dispensed solution or suspension. Cooling does not assume a change in state from liquid to solid and strictly should be used to describe reducing temperature during the initial stage of freeze-drying. Freezing refers to the abrupt phase change when water freezes as ice. Except for very complex biomolecules or cold sensitive cells, cooling in the absence of freezing (chilling) is generally not damaging to biomaterials. When solutions or suspensions are frozen, they may cool appreciably below their measured freezing point prior to ice formation, a phenomenon defined as super-cooling (undercooling or sub-cooling). The extent of super-cooling depends on cooling rate, sample composition and cleanliness, dispensed fill volume, container type, method of sample cooling, and so on. Even when a simple solution is repeatedly cooled or warmed, the onset and extent of super-cooling will vary from cycle to cycle. In the super-cooled state, while the composition of the solution remains unchanged, the cooled liquid is thermodynamically unstable and sensitive to ice formation. As the solution is cooled to lower temperatures, the probability of ice crystallization will correspondingly increase. For optimized freeze-drying, the intention should be to induce super-cooling in the suspension to encourage uniform cooling and freezing throughout the sample contents.

Sample freezing may be defined as the abrupt conversion of the suspension into a mixture of ice and solute concentrate. Freezing is a two-step process during which water initially nucleates, followed by the growth of the ice crystals that pervade the solute phase resulting in a mixture of ice and solute concentrate. Under typical processing conditions, ice nucleates heterogeneously around microscopic particles within the suspension and is encouraged by reducing temperature and agitating the super-cooled suspension to increase the probability of contact between nucleating foci and water clusters. Nucleation depends on the number and physical nature of particulate impurities within the suspension or solution. Ice is a particularly effective nucleation focus and cryobiologists may deliberately seed samples with ice to induce nucleation. Other effective ice nucleators include glass shards and specifically formulated nucleation promoters. Whereas nucleation aids can be added to experimental systems, deliberate attempts to add ice inducers to pharmaceutical materials would be at variance with Good Pharmaceutical Manufacturing Practice.

In contrast to nucleation, ice growth (proliferation) is encouraged by raising the temperature, thereby decreasing the suspension viscosity. Ice nucleation and proliferation are inhibited at temperatures below the glass transition temperature (Tg′), whereas above the melting temperature (Tm) the suspension or solution will melt. The consequences and measurements of these parameters are important elements in the formulation exercise.

To facilitate the sublimation of water vapor from the drying mass, the ice crystals should be large, wide, and contiguous, extending from the product base toward its surface, thereby providing an optimized structure for vapor migration. Crystal structures commonly observed during freeze-drying when solutions are frozen in trays or vials include dendritic structuring, where the ice crystal branches continuously from the nucleating focus and the spherulite form, and where sub-branching is discouraged because the solution viscosity is high, or fast rates of cooling are used.

Cooling or freezing rates are defined as slow (suboptimal), rapid (super-optimal), or optimal as assessed by criteria such as post-freezing cell survival or biopolymer activity, and is ambiguous unless conditions are more precisely defined. Cooling rates may be defined in terms of:
1. The rate at which the shelf temperature is cooled per unit time.
2. The rate at which the solution cools per unit time.
3. The depth of liquid within the vial (in mm) which cools per unit time.

The shelf-cooling rate is the simplest parameter to control and programmed rates of cooling are standard options on research and production freeze-dryers. Because shelf temperature and product responses are not identical, defining shelf-cooling rate will not fully define product behavior. Although we are concerned with the cooling rate achievable within each vial, this parameter is less easy to monitor compared with shelf cooling, and freeze-drying cycles generally are controlled by programmed shelf cooling rather than feedback control from the sample. Cooling rates of the product/cell suspension will vary considerably from vial and throughout the sample within the vial and, consequently, measuring the temperature of vial contents at a fixed position will give only an approximation of the sample temperature variation.

Observing the freezing pattern of a number of vials arranged on a shelf will demonstrate that while the contents of some vials will freeze slowly from the vial base, neighboring vials may remain unfrozen and supercool appreciably before freezing instantly. This random freezing pattern will reflect differences in ice structure from vial to vial and translated into different drying geometries from sample-to-sample vials. In summary, freezing patterns will be related to:
1. The ice forming potential within each vial.
2. The relative position of the vial on the shelf causing exposure of individual vials to cold or hot spots.
3. Edge effects where samples in vials on the periphery of each shelf will be subjected to heat transmitted through the chamber walls or door.
4. The insertion of temperature into the sample, which will induce ice crystallization.
5. The evolution of latent heat as samples freeze, which will tend to warm adjacent containers.
6. Variations in container base geometry, which may impede thermal contact between sample and shelf.

The ice and solute crystal structure resulting from sample freeze has a major impact on subsequent freeze-drying behavior, encouraging the sample to dry efficiently or with defects such as melt or collapse depending on freezing rate used. The preferred ice structure comprising large contiguous ice crystals is induced by freezing the sample at a slow rate of c. 0.2-1.0° C./min. Slow cooling will also induce the crystallization of solutes reluctant to crystallize when faster rates of cooling are used. However, a slow rate of cooling may exacerbate the development of a surface skin, which inhibits sublimation efficiency. Slow cooling can also inactivate a bio-product by prolonging sample exposure to the solute concentrate biomolecules. However, a fast rate of cooling can result in the formation of numerous, small, randomly orientated ice crystals embedded in an amorphous solute matrix, which may be difficult to freeze-dry. Complicating the choice of freezing regimes is the fact that the optimal cooling rate cannot be sustained where the sample fill depth exceeds 10 mm. In short, defining cooling rates often requires a compromise in sample requirements.

### Ice Structure and Freeze Consolidation

A period of consolidation (defined as the hold time), is necessary at the end of sample cooling to ensure that all the vial contents in the sample batch have frozen adequately, although excessive hold times will increase the time of sample freeze and impact on the overall cycle time. It is a fallacy to assume that the ice structure induced remains unchanged during this consolidation period and an ice structure comprising a large number of small ice crystals, induced by rapid cooling, is thermodynamically less stable than an ice structure comprising fewer, larger crystals. The thermodynamic equilibrium can be maintained by recrystallization of ice from small-to-large crystals, a process termed grain growth. Although ice structure changes take place randomly from vial to vial, the hold period is a major factor in ice recrystallization resulting in significant variation in crystal structure and subsequent sublimation efficiency from sample to sample the longer the hold period is employed.

As an alternative to increasing the length of the hold time to encourage ice recrystallization, a more controlled and time-efficient method of inducing recrystallization is to heat anneal the frozen sample. Essentially heat annealing is achieved by:
1. Cooling the product-to-freeze solution water and crystallized solutes.
2. Raising the product temperature during the freezing stage to recrystallize ice from a small to a large ice crystal matrix. (Note, that this warming phase may also crystallize solutes that are reluctant to crystallize by cooling.)
3. Cooling the product to terminal hold temperature prior to chamber evacuation.

Heat annealing (also defined as tempering) is particularly useful to:
1. Convert an ice structure to a crystalline form, which improves sublimation efficiency.
2. Crystallizing solutes that are reluctant to crystallize during cooling.
3. Provide a more uniform, dry structure throughout the product batch.
4. Integrated with rapid cooling, heat annealing may minimize the development of a surface skin on the sample thereby facilitating sublimation.
5. Because heat annealing induces a more porous cake structure with improved drying efficiency, a lower dried sample moisture content may be achieved, with improved solubility.

Although heat annealing will increase the length of the freezing stage of the cycle, overall freeze-drying cycle times may be significantly reduced because of improvements in drying efficiency resulting from heat annealing. Care should be exercised when selecting temperatures and hold times for heat annealing, particularly when defining the upper temperature for sample warming. Subjecting a labile product, such as a vaccine, to temperatures above the eutectic temperature will expose the sample to hypertonic solution concentrates as the sample partially melts, which can damage sensitive biomolecules.

Amorphous materials do not have a eutectic point, but they do have a critical point, below which the product must be maintained to prevent melt-back or collapse during primary and secondary drying.

### Freezing Solute Behavior

Regardless of the precise freezing pattern, the formation of ice will concentrate the remaining solution within the container. As the proportion of ice increases within the mixture, solute concentration will correspondingly increase. In the case of an aqueous 1% (w/v) saline solution, this concentration effect will be considerable, increasing to approximately 30% (w/v) just prior to freezing, and damage to biomolecules results as a consequence of solute concentration exposure rather than direct damage by ice crystals. The behavior of the solute(s) within the solute concentrate depends on the nature, concentration, cooling rate, and interactions between individual solutes present in the medium and forms the basis for experimental review during a formulation development exercise.

Overall, four patterns of solute response are observed during freeze-drying:
1. Solute crystallizes readily, regardless of cooling rate or freezing conditions, to form a mixture of ice and solute crystals (this behavior is termed eutectic freezing).
2. Solute crystallizes, but only when the solution is subjected to a slow rate of cooling.
3. Solute crystallizes only after the solution has been heat annealed.
4. Solute fails to crystallize regardless of cooling rate or regime adopted, and solute remains associated with unfrozen water as a metastable amorphous mass or glass.

For a crystallizing solute, the eutectic point is the lowest temperature in a system in which a residual liquid phase and solid phase are in equilibrium. Above the eutectic point, ice and solute concentrate persist, whereas below the eutectic point, a mixture of ice and solute crystals is formed. Eutectic temperatures for aqueous solutions containing crystallizing salts are characteristic for each solute and are significantly below the freezing point of water (for example, eutectic temperature for sodium chloride =−21.4° C.). Exposing cells or proteins for prolonged periods to a eutectic solution comprising hypertonic salt concentrations can cause damage by plasmolysis or precipitation by “salting out”. The eutectic zone is the range of temperatures encompassing all the eutectic temperatures within the system. For a two-part water/solute system, the eutectic temperature is a discrete, quantifiable temperature in contrast to multi-solute systems where a eutectic zone may be observed that represents a range of temperatures where the minimum eutectic temperature is lower than that of any individual eutectic temperatures in the medium.

Typical freeze-dried vaccine formulations fail to crystallize completely when cooled, and a proportion of the solutes in the sample persist as an amorphous, non-crystalline, glass-like. When exposed to temperatures above their glass transition (Tg') or collapse temperature (Tc), these samples may warm during sublimation causing the amorphous mass to soften so that freeze-drying progresses with sample collapse to form a sticky, structure-less residue within the vial. Less severe collapse will result in the formation of a shrunken, distorted, or split cake. Collapsed cakes are not only cosmetically unacceptable but may be poorly soluble, exhibit reduced activity, or compromised shelf stability. Collapse may be exacerbated by the formation of a surface skin, which impedes vapor migration from the drying structure. To avoid sample collapse, it is necessary to maintain the sublimation interface below Tg′ or Tc throughout primary drying and to include excipients in the formulation, which reduce the severity of collapse. It is therefore essential to characterize formulations experimentally during the process development program. Although collapse may cause operational difficulties during freeze-drying, the induction and maintenance of the amorphous state may be essential for protecting labile biomolecules during freezing, drying, and storage.

For clarity, it is usual to separate the drying cycle into primary drying (the sublimation stage) and secondary drying or desorption.

### Stage 3—Primary Drying

The first step in the drying cycle is defined as primary drying and represents the stage where ice, which constitutes between 70 and 90% of the sample moisture, is converted into water vapor. Sublimation is a relatively efficient process although the precise length of primary drying will vary depending on the sample formulation, cake depth, and so on. During primary drying, the sample dries as a discrete boundary (the sublimation interface), which recedes through the sample from surface to base as drying progresses.

During the primary drying phase, the pressure is lowered (to the range of a few millibars), and enough heat is supplied to the material for the water to sublime. The amount of necessary heat can be calculated using the sublimating molecules' latent heat of sublimation. In this initial drying phase, up to 95% of the water in the material is sublimated. This phase may be slow (can be several days in the industry), because, if too much heat is added, the material's structure could be altered.

In this phase, pressure is controlled through the application of partial vacuum. The vacuum speeds up the sublimation, making it useful as a deliberate drying process. Furthermore, a cold condenser chamber and/or condenser plates provide a surface(s) for the water vapor to re-solidify onto. This condenser plays no role in keeping the material frozen; rather, it prevents water vapor from reaching the vacuum pump, which could degrade the pump's performance. Condenser temperatures are typically below −50° C. (−60° F.).

It is important to note that, in this range of pressure, the heat is brought mainly by conduction or radiation; the convection effect is negligible, due to the low air density.

Variously described as the drying front, freeze-drying front, and so on, macroscopically the sublimation interface can be observed as a discrete boundary that moves through the frozen sample to form an increasingly deeper layer of dried sample above the frozen sample. Heat is conducted from the shelf through the vial base and the frozen sample layer to the sublimation front where ice is converted into water vapor. Several consequences result from this progressive recession of the sublimation front through the dry layer, which include:
1. The maintenance of the frozen zone at a low temperature because of sublimation cooling.
2. An increase in the resistance to vapor migration and a decrease in sublimation rate as the dry layer increases in thickness.
3. Because the sublimation interface represents a zone representing maximum change of sample temperature and moisture content, the interface represents the zone over which structural softening or collapse is likely to occur.
4. Water migrating from the sublimation front can reabsorb into the dried material above the sublimation interface.

Although the sublimation interface is defined as a discrete boundary, this is true only for ideal eutectic formulations, where ice crystals are large, open, and contiguous with each other. For typical amorphous formulations, such as vaccines, the sublimation front is much broader and comprises individual ice crystals imbedded in the amorphous phase. Under these conditions, although ice sublimes within the isolated crystals, the water vapor must diffuse through the amorphous phase (which is itself progressively drying) until it can migrate freely from the drying sample matrix. Under these conditions, sublimation rates are much lower than those anticipated from data derived using eutectic model systems. Complicating a precise prediction of sublimation rate is the fact that fractures in the dry cake between the ice crystals can improve drying efficiency. All of these factors, including system impedances caused by the development of a surface skin on the sample, have to be considered during sample formulation and cycle development programs.

Notwithstanding these complications in precisely defining primary drying, sublimation is nevertheless a relatively efficient process and conditions used for primary drying include the use of shelf temperatures high enough to accelerate sublimation without comprising sample quality by inducing collapse or melt, combined with high system pressures designed to optimize heat conduction from shelf into product. Removing the product when sublimation has been judged as complete will provide a vaccine which appears dry but which displays a high-moisture content that is invariably too high (7-10%) to provide long-term storage stability, and the drying cycle is extended to remove additional moisture by desorption or secondary drying.

### Stage 4—Secondary Drying

In contrast to primary drying, which is a dynamic process associated with high vapor flow rates, secondary drying is much less efficient with secondary drying times representing 30-40% of the total process time but only removing 5-10% of the total sample moisture. Under secondary drying conditions, the sample approaches steady-state conditions where moisture is desorbed or absorbed from or into the sample in response to relative humidity and shelf temperatures. Desorption is favored by increasing shelf temperature, using high-vacuum conditions in the chamber, thereby reducing the system vapor pressure or relative humidity. Conversely, when the shelf temperature is reduced and the vapor pressure in the system increased by warming the condenser, dried samples will reabsorb moisture and exhibit an increase in moisture content. Although sample collapse during secondary drying is generally less likely than collapse during primary drying, it is possible to induce collapse in the dried matrix by exposing the sample to temperature above its glass transition temperature (Tg).

The secondary drying phase aims to remove unfrozen water molecules, since the ice was removed in the primary drying phase. This part of the freeze-drying process is governed by the material's adsorption isotherms. In this phase, the temperature is raised higher than in the primary drying phase, and can even be above 0° C., to break any physico-chemical interactions that have formed between the water molecules and the frozen material. Usually the pressure is also lowered in this stage to encourage desorption (typically in the range of microbars, or fractions of a pascal). However, there are products that benefit from increased pressure as well.

After the freeze-drying process is complete, the vacuum is usually broken with an inert gas, such as nitrogen, before the material is sealed.

At the end of the operation, the final residual water content in the product is extremely low, around 1% to 4%.

### Sample Damage During Freeze-Drying

Damage to a freeze-dried product may occur:
1. When the solution is cooled (described as cold or chill shock).
2. During freezing as the ice forms and the unfrozen solute phase concentrates.
3. During drying, particularly when the sample collapses as drying progresses.
4. By protein polymerization when high shelf temperature are used for secondary drying.
5. During drying and storage because of damage by reactive gases, such as oxygen, and it is important to appreciate that even in a vacuum, sufficient gas molecules will be present in the sealed sample to cause inactivation.
6. During storage by free radical damage or Maillard reactions.
7. During reconstitution, particularly if the sample is poorly soluble.

### Chill Damage (Cold Shock)

Reducing temperature in the absence of ice formation is generally not damaging to biomolecules or live organisms, although sensitive biopolymers may be damaged by cold shock.

### Freezing Damage

Reducing temperature in the presence of ice formation is the first major stress imposed on a biomolecule. Direct damage by ice is not generally damaging except when living cells are frozen at very fast rates, which may induce the formation of intracellular ice within the cell. Biomolecules are more likely to be damaged by an increase in solute concentration as ice forms. We have described how bacteria can remain fully viable when cooled to −18° C. in the absence of ice formation, but when frozen to this temperature, viability was reduced to 60%. Freezing will result in:
1. Ice formation.
2. A rise in solute concentration (this effect can be appreciable and a 1% solution of sodium chloride will increase to 30% by freeze concentration as ice forms).
3. Changes in solution tonicity.
4. Concentration of all solutes, including cells and biomolecules that are encouraged to aggregate.
5. An increase in solute concentration that may result in “salting out” of protein molecules.
6. Differential crystallization of individual buffer salts resulting in marked changes in solution pH as the solution freezes.
7. Concentration of potentially toxic impurities above a toxic threshold sufficient for the impurities to become toxic.
8. Disruption of sulfur bonds.
9. Generation of anaerobic conditions as freezing progresses.

### Cryopreservation—Preservation of Cells and Tissues

Lowering the temperature of biological material reduces the rate of metabolism until all internal water is frozen and no further biochemical reactions occur [7]. This is quite often lethal and at the very least causes cellular injury. Although fungi are quite often resistant to ice-induced damage, cooling must be controlled to achieve optimum survival. The avoidance of intracellular ice and the reduction of solution, or concentration, effects are necessary [8]. Little metabolic activity takes place less than −70° C. However, recrystallization of ice can occur at temperatures greater than −139° C. [9] and this can cause structural damage during storage. Consequently, cooling protocols have to be carefully designed for cells in order to inflict the least damage possible. The cryopreservation of microfungi at the ultra-low temperature of −196° C. in liquid nitrogen or the vapor above is currently regarded as the best method of preservation [20,10,11]. It can be widely applied to sporulating and non-sporulating cultures. Initial work with fungi was undertaken by Hwang [12] who employed a method designed for freezing avian spermatozoa [13]. Similar methods have been used successfully [11,14,15]. Provided adequate care is taken during freezing and thawing, the culture will not undergo any change either phenotypically or genotypically. Optimization of the technique for individual strains has enabled the preservation of organisms that have previously been recalcitrant to successful freezing [14]. The choice of cryoprotectant is a matter of experience and varies according to the organism. Glycerol gives very satisfactory results but requires time to penetrate the organism; some fungi are damaged by this prolonged incubation step. Dimethyl sulfoxide penetrates rapidly and is often more satisfactory [16,17], but is often toxic to sensitive organisms. Sugars and large molecular substances such as polyvinylpyrrolidone [18,19] (FIG. 2) have been used but, in general, have been found to be less effective.

### Preservation of Cells

The basic cryobiological knowledge reviewed here has made it possible to develop effective methods for the preservation of a very wide range of cells, and these have found widespread applications in biology and medicine. Examples include the long-term preservation of spermatozoa of many species, including cattle, laboratory animals, and man, very early embryos and ova, red and white blood cells, hemopoietic stem cells, tissue culture cells, and so on (see following chapters in this volume). For each type of cell there is a set of conditions that is optimal for preservation, determined by the interaction of the particular properties of the cell in question with the cryobiological factors that have been discussed. If the characteristics of the cell are known, it is usually possible to predict with reasonable precision the conditions that will provide effective cryopreservation (FIG. 3).

### Preservation of Multicellular Systems

The situation becomes much more difficult when we move from single cells to complex multicellular systems. Cell survival is still required, of course, but tissues and organs contain a heterogeneous collection of cells, which may have quite different optimum requirements for preservation, unlike the situation in cell preservation where one is usually dealing with a single type of cell. Yet it is necessary to find a method that will secure adequate survival of all the cells that are important for the function of that tissue. Fortunately, the use of high concentrations of cryoprotectant results in a flattening of the bell-shaped survival curve and a broadening of its peak: with sufficiently high concentrations of cryoprotectant it is possible to secure overlapping survival curves for many different cells. Another problem is that it is not sufficient to obtain high levels of survival for the various types of cell that are present in tissues and organs; it is also imperative to avoid damage to important extracellular structures and to retain normal interconnections between the cells and their attachments to basement membranes [21]. Ice that forms outside the cells when a cell suspension is frozen is outside the system that it is desired to preserve, and it can damage the cells only by indirect means (solution effects) or by exerting a shear or compressive force on them externally. The situation is quite different for organized tissues; here, extracellular ice is still within the system that is to be preserved and can disrupt the structure of the tissue directly. The first evidence of such an effect was provided by Taylor and Pegg [22] when they showed that smooth muscle, frozen to −21° C. by cooling at 2° C./min in the presence of 2.56 M dimethyl sulfoxide was functionally damaged, whereas exposure to the solution conditions produced by freezing that solution to that temperature, at the same temperature, was innocuous. Structural studies using freeze substitution showed that ice formed within the muscle bundles [23]. If cooling was slowed to 0.3° C./min, freezing produced less damage and ice was shown to form only between the muscle bundles. This showed that extracellular ice damaged this tissue, but the extent of such damage was dependent on the site at which the ice formed. Damaging effects of extracellular ice have also been demonstrated in kidneys and livers, where it has been shown to cause rupture of the capillaries. Rubinsky and Pegg [24] have proposed a mechanism for this effect; ice forms within the vessel lumens, drawing in water from the surrounding tissue until the volume of intraluminal ice exceeds the elastic capacity of vessel and rupture ensues. In organs and tissues that require an intact vasculature for function, vascular rupture is lethal, even if many cells survive, and this mechanism provides the major barrier to effective cryopreservation of such systems. The avoidance of freezing, or at least limitation of the amount of ice to very small quantities in the least susceptible locations, seems to be the only way to avoid this problem. Attempts to cryopreserve complex multicellular systems simply by adapting techniques from single-cell systems have generally been unrewarding. In the medical field, the situation may be more favorable with tissues that can be transplanted without revascularization; it all depends on the precise requirements for surgical acceptability. For example, the primary requirement for heart valve grafts is that the collagen structure is intact, and it is unclear whether the survival of donor fibroblasts has any useful effect. Similarly, human skin can be cryopreserved by methods similar to those used for cell suspensions and will then retain significant numbers of viable keratinocytes, although it is questionable whether these influence the clinical results when skin grafts are used as a temporary covering on seriously burned patients. For other tissues, such as small elastic arteries, satisfactory methods have only been developed relatively recently [25]. For corneas and cartilage and for whole vascularized organs there are no effective methods.

### Cryopreservation of Plant Cell Suspensions

The cryopreservation of dedifferentiated cells, grown in suspension culture, is one of the portfolio of techniques employed for the long-term conservation of higher plant germplasm. Suspension cultures are also important in biotechnology, particularly in transformation studies and for the production of specific metabolites, and, here, there is also a pressing need for genetically stable, long-term storage of cell lines. Cryopreservation of suspension cell cultures can be exploited by either slow, or rapid, cooling techniques. During slow cooling the extracellular solutions are nucleated and the cells cryodehydrate during controlled cooling as a consequence of extracellular ice, to the point where their intracellular fluids will vitrify on subsequent transfer to liquid nitrogen. In the rapid cooling protocols, the cells are prepared by extreme osmotic dehydration, with cryoprotection, before plunging the samples directly into liquid nitrogen to achieve vitrification. Extensive success has been achieved with both techniques but rapid cooling is, currently, widely favored because of its simplicity.

The consequences and advantages of cryoconservation and its application to plant suspension cultures are well documented [27-31]. The first generation of techniques, still current and widely employed, were based on a protocol reliant on relatively slow-cooling rates, at or near 1° C./min [27-32] and a cryoprotectant mixture containing dimethyl sulfoxide (DMSO), glycerol, and sucrose. For many culture types, acceptable levels of success with this basic method depended on empirical optimization of:
1. The duration and nature of any pretreatments used to take advantage of natural cell adaptation.
2. The type, mixture, and concentration of cryoprotectants.
3. The incubation time in the protectant solution.
4. Precise cooling rate.
5. The duration of any necessary subzero-holding temperatures during the cooling protocol that increased cryodehydration.
6. The recovery conditions.

Typically, suspensions are concentrated from the growth medium at a point in the culture cycle where the proportion of small, more densely cytoplasmic cells is at a maximum, with these being viewed as the likely survivors of the freezing protocol. Dependent on the particular genotype, the suspensions may benefit from pre-growth with enhanced concentrations of an osmotic such as mannitol or sorbitol and proline and DMSO have also been used in pretreatment [26,28,33,34]. This pre-growth takes advantage of any natural, adaptive responses that result from osmotic stress and/or activation of the synthesis of natural protectants [35]. Pre-growth at low temperature may also be beneficial [36,37]. Thereafter, the cells are exposed to a cryoprotectant solution containing, in the original protocol, DMSO, glycerol, and sucrose at final concentrations of 0.5, 0.5, and 1 M, respectively [28,38]. The optimum concentrations will vary with circumstance, and empirical optimization is likely to be needed. Other protectant compounds used include proline and polyethylene glycol. Once pretreated, volumes of cell suspension of less than 2 mL are cooled at a rate typically between 0.5 and 2.0° C./min to −40° C., before plunging into liquid nitrogen. At these slow-cooling rates, the unfrozen suspension cells become embedded in ice following nucleation of the extracellular solution, resulting in ongoing cryodehydration [30,31]. As temperature decreases, the dehydration continues and intracellular solutions become increasingly concentrated and viscous. A point is reached where the residual intracellular solutions will vitrify when the cell sample is plunged directly into liquid nitrogen. A holding period at an appropriate subzero temperature may be required to achieve a sufficient level of dehydration. This pattern of cooling, followed by the plunge to ultra-low temperature, provides the classic “two-step” freezing protocol. The expectation is that, with rapid rewarming to avoid intracellular ice crystallization, a proportion of the cells in suspension will retain organization and viability following thawing [28,38,39].

A second major step in the cryopreservation of plant cell suspensions is seen in the more recent proliferation of vitrification techniques, whereby cells can be cooled rapidly to ultra-low temperatures avoiding the phase transition from liquid water to crystalline ice and, instead, producing an amorphous glass [39]. Two mechanisms are at work in the successful vitrification protocol, the first being extensive cytoplasmic dehydration resulting from high concentrations of essentially non-penetrating protectants, e.g., glycerol at 30% (w/v) in the extracellular medium [39]. The second is the increase in viscosity of the cytoplasmic solution that follows from the penetration of high concentrations of protectant, such as DMSO, included in the added Vitrification medium into the cells.

Following treatment with vitrification medium, both the extra- and intracellular solutions are radically modified as a consequence of osmotic dehydration and loading with high concentrations of cryoprotectants. The cells that survive are those that become sufficiently dehydrated so that their intracellular solutions form a stable glass when, with rapid cooling by direct immersion of the sample in liquid nitrogen, they pass the appropriate glass transition point. Typically, the cooling rates employed are in excess of 200° C./min. The extracellular solutions must undergo a similar transition. For the widely used plant vitrification solution PVS2 containing high levels of glycerol, ethylene glycol, and DMSO, this occurs in the region of −115° C. [39]. The presence of a glass provides stability in the system to be preserved, but successful recovery depends on a thawing procedure that transforms the aqueous glass phase directly to a liquid without the intervention of crystalline material, which can only be achieved by rapid rewarming. Precise rates are not usually quoted for this part of published protocols, perhaps because of the difficulties in making appropriately cited, accurate measurements. However, immersion of 2-mL cryo-vials, foil packets, or straws in water at 40° C. for 1-2 min is widely reported [37,39,40]. On occasion, the vitrification protocol can be modified to include an encapsulation stage, whereby the cells are embedded in alginate beads prior to the vitrification procedures [41].

### Cryopreservation of Shoot Tips and Meristems

Recent advances in shoot-tip cryopreservation have been significant, this is largely because of: (1) development of vitrification-based cryoprotection protocols, (2) refinements in tissue culture practices, (3) identification of critical points in cryopreservation technology transfer, and (4) the wider uptake and validation of cryostorage technologies in international germplasm repositories. There still remain some genotypes intractable to cryogenic storage, and fundamental research is progressively facilitating the identification of decisive factors in recalcitrance, with a view to aiding storage protocol development. With these issues in mind, this chapter will report the routine storage and investigative methodologies currently applied to shoot cryopreservation. Generic cryopreservation protocols will be described and Ribes (currants) are used as an exemplar, as this genus has been studied in detail with regard to critical point thermal analysis, protocol validation, and technology transfer to germplasm repositories.

There are two main approaches to cryopreservation based on cryoprotective modality. The first is termed traditional, controlled rate, or two-step freezing and involves the application of single or combined mixtures of colligative cryoprotectants, sometimes in conjunction with osmotic additives. Controlled cooling of tissues to an intermediate freezing temperature causes a water vapor deficit to be created between the inside and outside of the cell initiated by the preferential formation (termed “seeding” or ice nucleation) of extracellular ice. Thus, intracellular water moves across the plasmalemma and cellular dehydration results; this process is called equilibrium freezing. Under optimum freezing rates this is a cryoprotective process, as the amount of water available for intracellular ice formation is reduced. However, excessive dehydration can lead to colligative damage owing to the harmful concentration of solutes. Critical cryogenic factors in controlled-rate freezing are:
1. Cryoprotectant composition, the components of which must include a penetrating colligative cryoprotectant.
2. Cooling rate.
3. The control of, and point at which, ice nucleation occurs.
4. A “hold step” programmed as a fixed time at a fixed terminal transfer temperature. Usually at, or around, the point of homogeneous ice formation, which is −40° C.
5. Transfer to liquid nitrogen.

The second approach to plant cryopreservation is vitrification which usually comprises different permutations of chemical additive as well as vitrification, and encapsulation, osmotic-, evaporative-, and chemical (silica gel)-dehydration. The cryoprotective basis is the concentration of solutes to such an extent that cell viscosity becomes so high that on exposure to cryogenic temperatures, a glass, rather than ice, is formed. The process, termed vitrification, involves the creation of a metastable amorphous glassy state, characterized by the glass transition temperature, Tg, the thermal point at which a glass is formed. However, glasses can be thermally unstable and it is possible that de-vitrification occurs on cooling and rewarming (as this risks the formation of ice it is critical that glasses are stabilized). This can be achieved by the manipulation of dehydration and cryoprotective additives, particularly the inclusion of sugars and careful control of rewarming. Vitrification is a useful alternative to controlled-rate cooling and freezing, as it has the major advantage that tissues are directly immersed into liquid nitrogen, circumventing the need for expensive programmable freezing equipment. In addition to cryogenic factors, the development of successful cryopreservation protocols also depends on the physiological status of the tissues, and pre- and post-treatments are applied to maximize the ability of shoot tips to survive cryopreservation. Detailed information as to the theory and applications of plant cryopreservation can be found in a fundamental theory of cryopreservation by Mazur [42]; cryo-physics and instrumentation reviewed by Benson et al. [43]; historical perspectives and wider areas of in vitro plant conservation are highlighted by Benson [44,45]; and a comprehensive overview of the contemporary aspects of fundamental and applied cryobiology is detailed by Fuller et al. [46].

### Factors Effecting Dried Products

Freeze-dried vaccines should be formulated to minimize storage decay and should tolerate storage at ambient temperatures for distribution purposes. However, it is a fallacy to suppose that a freeze-dried product remains immune to damage during storage and factors which damage freeze-dried products include:
1. Temperature. Whereas a freeze-dried product is more shelf stable than its solution counterpart, freeze-dried materials are sensitive to thermal decay and will be influenced by storage temperature.
2. Moisture content.
3. Reactive gases.
4. Light.
5. Free radical damage.
6. Background nuclear radiation.
7. Specific chemical reactions including Maillard reactions.

The interrelationship between sample formulation, dried cake moisture cake, storage conditions, and glass transition temperature (Tg) are complex. In general terms, any physical distortion of the dry cake during storage will often result in a much more rapid loss of sample activity than predicted using the Arrhenius equation for reviewing similar samples.

### Properties of freeze-dried product

If a freeze-dried substance is sealed to prevent the reabsorption of moisture, the substance may be stored at room temperature without refrigeration, and be protected against spoilage for many years. Preservation is possible because the greatly reduced water content inhibits the action of microorganisms and enzymes that would normally spoil or degrade the substance.

Freeze-drying also causes less damage to the substance than other dehydration methods using higher temperatures. Freeze-drying does not usually cause shrinkage or toughening of the material being dried. In addition, flavors, smells and nutritional content generally remain unchanged, making the process popular for preserving food. However, water is not the only chemical capable of sublimation, and the loss of other volatile compounds such as acetic acid (vinegar) and alcohols can yield undesirable results.

Freeze-dried products can be rehydrated (reconstituted) much more quickly and easily because the process leaves microscopic pores. The ice crystals that sublimate, leaving gaps or pores in their place, create the pores. This is especially important when it comes to pharmaceutical uses. Freeze-drying can also be used to increase the shelf life of some pharmaceuticals for many years.

### BRIEF SUMMARY OF THE INVENTION

Main goal in herbal-, pharmaceutical-, and food-oriented industries is the initial preservation of biological material immediately at or after the vitality-state is altered or destroyed. The basic reason for this is to preserve all biologically active ingredients as-is in their native state and function with almost no alterations. Moreover, the methodology maintaining the state of preservation must be both effective on all bio-material manipulation stages and commercially viable—handling large amounts of biological material and maintaining perfect preservation over extended periods of time up to years after the vitality is interrupted or destroyed.

In opposite to the common commercial lyophilization practices where water crystallization is well sought approach and result in order to facilitate the fast evaporation, in our approach for optimal bio-chemical and structural preservation we employ rapid and instant deep freezing of the biological objects under conditions leading to residual water “glassification” (or so called vitrification—instant freezing without crystallization and volume increase) entirely outsourcing the crystallization followed by water evaporation, manipulations and encapsulation of the biological material as a commercial product without any treatments harmful for degrading the bio-chemical content nor cellular structure—leading to preparation of supplements as natural as the alive biological original obtained initially.

In an optimized setting, the method for preservation of biological substances is based upon the construction and utilization of volume-enclosed container-type vessels comprising deep-freezing fluids, solids and/or gases capable of both engulfing, and instantly freezing any biological material added into vessel, and maintaining deep-freeze temperatures (optimally—below minus−25 degree Celsius) during the bio-material collection, transportation and initial pre-manipulation storage process. Best instant freezing is achieved by collecting the biological material from the field and submersing it immediately into a vessel containing liquefied gas or mixture of gases including but not limited to nitrogen in amounts ensuring the instant engulfing of the entire biological material within the frozen gas molecules leading to immediate deep-freezing. It is essential for the initial freezing to employ frozen liquids and/or liquefied gases at lowest possible temperatures (preferably from minus−180 and minus−80 to minus−30 degree Celsius) in order to best manage achieving water glassification (vitrification) state for best bio-material preservation. Further temperature increase up to minus−20 degree Celsius can be tolerated since it can increase water sublimation with no interference on the glassification (vitrification) state—i.e. no water crystals will be formed and no cells rupture will occur due to volume expansion once a rapid deep-freezing is achieved at temperatures preferably below −35° C. and the temperature after-on is maintained below −20° C.

When any partitioning of the biological materials is desired (such as separating roots, stems, leafs and/or flowers from an intact plant or else) it is preferable that such manipulations are performed immediately upon the collection of the biological material before the deep-freezing is employed since it is highly inconvenient and costly to perform separation manipulations further on while maintaining a frozen conditions. For the purpose of such a practical setup, the deep-freezing collection container is separated on sections allowing for separate collection and freezing of each part of the biological material unless entirely separate deep-freezing collection vessels are employed when needed. In cases when a cross-contamination is anticipated and is essentially not desired, employing separate deep-freezing vessels (containers) is a preferred choice.

After the initial rapid deep-freezing the biological material is transported and stored in freezing containers maintaining temperatures below −20° C. The construction type and the type of the system maintaining the freezing temperatures is not of importance, however the presence of inert gaseous atmosphere (such as Nitrogen, Argonne, Neon, Xenon) is highly desirable since it prevents from oxidation of the biological components—especially if the material has been mechanically disrupted to a higher degree prior or after the deep-freezing.

Commercial manipulation of the biological material starts with reducing the volume of the material for optimizing the drying process. This involves procedures for bio-material disruption such as chopping, cutting, grinding, mulching, etc.—all performed in frozen conditions not increasing −20° C. and resulting in downsizing the biological material to a particles of a preferred size. This step is necessary for two main reasons: first, for down-sizing the entire volume of the total bio-material amount since the following drying step requires minimizing the volume of the drying vessel for economical reasons and switching from submersible freezing (the bio-material is mixed and/or in molecular contact) with the freezing substance) to an external freezing (the bio-material is in contact only with the vessel walls and the gases inside it, and the coolant is outside the vessel walls); second, the size-reduction of the biomaterial particles increases the total contact-surface of the particles, which by itself promotes an increased efficiency of the drying and water evaporation from the bio-material.

The drying of the biomaterial is performed preferably by vacuumization of the storage vessel of a limited total volume while maintaining temperature below −20° C. Were convenient, the drying chamber may be equipped with a mixing device to promote equal surface access for all bio-material particles subjected to drying. The drying chamber is usually constructed with glass- and/or metal walls and parts for best freeze conductivity and mechanical strength. Were reasonable—optical and electro-chemical sensors may be employed for monitoring the processes of bio-material disruption, drying and other manipulations. A water sublimation and removal from the bio-material up to 85-95% completes the initial step of drying.

The secondary drying of the bio-material is usually employed for maximum preservation and increased shelf-life of the final commercial products. The secondary drying is performed after the removal of 90% of the water-content from the bio-material—by gradually increasing the temperature within the drying chamber. The preferable time of the gradual increase and the temperature of the secondary drying vary depending upon the total amount and size of the bio-material particles, the type—and size of the drying chamber, and the construction type of the freezing device. In general, the higher is the water-content of the bio-material—the lower must be the drying temperature with or without vacuumization of the drying chamber a vacuumization; when the residual water becomes below 5% (w/w)—the chamber temperature may be increased to 0° C. and above, preferably to up to 40° C. in a gradual manner usually (but not—obligatory) by a step of −5° C. per hour per kilogram of bio-material. Once the water-content within the bio-material falls below 1-2%, the secondary drying is accomplished. For certain highly-hygroscopic bio-materials, the temperature of the secondary drying may be increased up to 60-75° C. with certain considerations that some enzymes and other bio-components (such as aromatic oils and chemicals) are not stable and/or evaporate and increased temperatures. The main principle during the bio-material drying is to maintain the drying at lowest possible temperature while achieving 97-99.5% of water removal.

After the bio-material is dried it must be stored at low temperatures and humidity that prevent from water condensation. A preferred temperature range is 5-15° C. Large stock-volumes of bio-material may be stored in large buckets of a sizes 5-20-50 gallons filled with inert-gas atmosphere to prevent bio-components' oxidation. The commercial-size containers and packages should be stored at the same conditions.

The dried bio-material can be processed for mixing, combining, aliquoting, encapsulating, etc. after primary drying (at approximately 85-95% of water removal) and/or after secondary drying (at approximately 95-99.5% of water removal). Often manipulating the bio-material at low-temperatures after primary drying is more preferable option since maintaining extremely low humidity levels (below 5%) required after the secondary drying is very challenging during bio-material's manipulation. Therefore only the encapsulation procedures and vial-filling have to be performed after the secondary drying and at humidity level preferable below 1-2%.

All additional manipulations and storage of the bio-material are performed preferably at inert atmosphere, relatively low ambient temperature (not increasing 15-20° C.) and minimal humidity. For best preservation of the final product, a final-step vacuumization of the storage vials may be performed and introduction of inert atmosphere (Nitrogen, Argon, etc.) may be desired before sealing hermetically the packing vials.

### BRIEF DESCRIPTION OF THE DRAWINGS

FIG. 1: In a typical phase diagram, the boundary between gas (G) and liquid (L) runs from the triple point (the 3-line cross) to the critical point (black dot). “S” indicate the solid-state. Freeze-drying (black arrow) brings the system around the triple point, avoiding the direct liquid-gas transition seen in ordinary drying time (light-grey arrow).

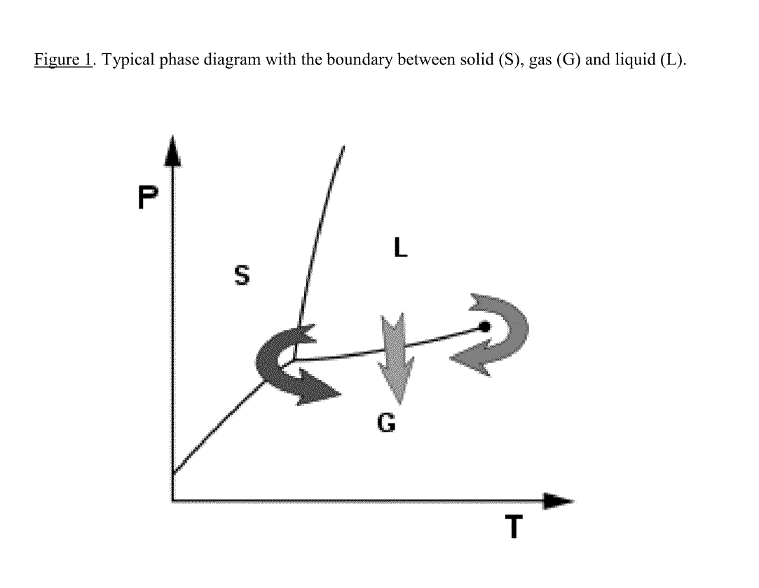

FIG. 2: Diagram constructed from data by Luyet [1] showing the time- and temperature dependence of nucleation and ice crystal growth in a thin film of a 50% (w/v) solution of polyvinylpyrrolidone. The arrows indicate cooling trajectories that avoid nucleation (300° C./s), nucleate without crystal growth (80° C./s) and produce ice crystals (20° C./s).

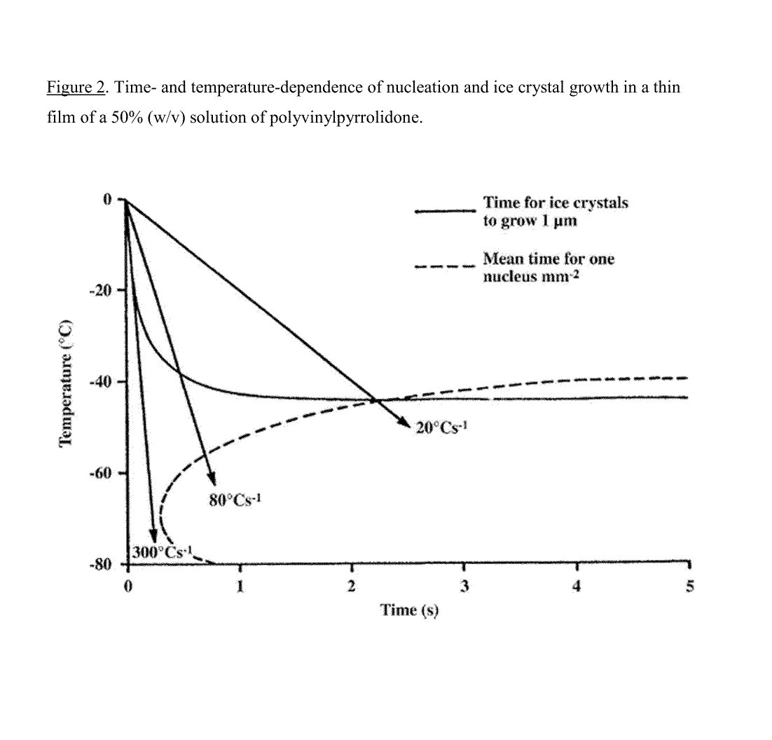

FIG. 3: The survival of three types of cells plotted against cooling rate and correlated with the observed occurrence of intracellular freezing.

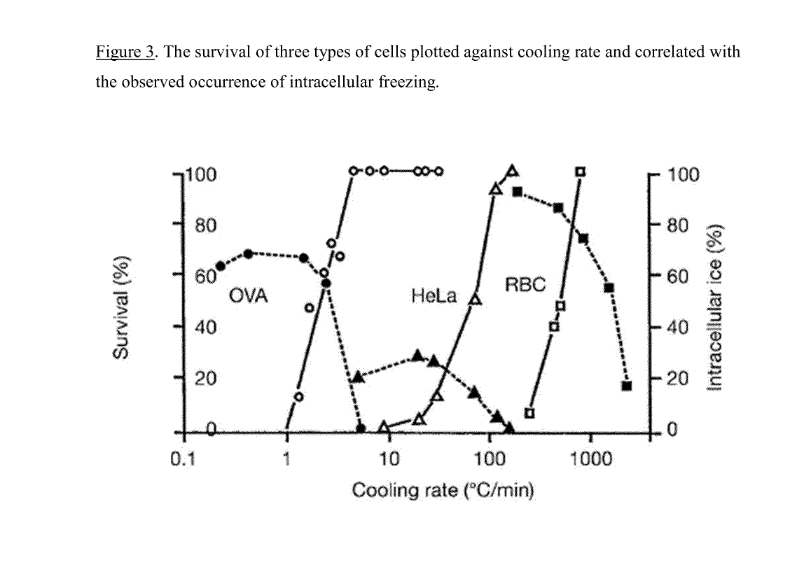

### DETAILED DESCRIPTION

The present invention will be described in further detail herein below.

Preservation of plans and fungi by drying has evolved along with the evolution of humanity. During the last century, however, the commercialization and the development of food and supplements industries had let to dramatic progress in implementing varieties of methods and technologies advancing the lyophilization method for preservation and drying of biological material and substances because of its efficiency and commercial viability. While some methods employing liquid nitrogen had been developed for laboratory isolation of biological substances such as RNA, DNA and proteins from limited amounts (of a gram-scale) of raw biological material and for storage with the addition of antifreeze additives, the commercial approaches and technologies have been employing entirely opposite principle of contact-freezing and lyophilization promoting the formation of ice crystals and cell disruption followed by material heating for water evaporation as a way to facilitate more economical preparation of frozen and dry nutrition goods.

The exploitation of instant deep-freezing with water glassification (vitrification) followed by cold lyophilization, however, is much more preferable choice for the preparation of herbal and pharmaceutical remedies. The availability of liquefied and solidified gases (such as nitrogen and carbon dioxide) at low prices and the price increase of herbal and supplement remedies had made the submersion-lyophilization commercially viable approach—especially because for the herbal industry there is no need to lyophilize as extremely large amounts of bio-material as it is needed in the food industry. The quality of preservation of biological material becomes of much higher importance in herbal and pharmaceutical industries making the submersion lyophilization a preferred choice.

The method of this invention is dedicated for commercial preparation and preservation of plants, funguses (fungi), lichens and/or algae, entire plant-, fungus-, lichen- and/or algae material, parts of entire plant-, fungus-, lichen- and/or algae species and/or any desired combinations of different biological species or parts of them, including but not limited to those serving herbal, medicinal and/or supplemental properties and purposes. It is not intended for implementation on biological material from animal-, bacterial- and viral origin nor purposes other than commercial ones thereby excluding direct laboratory- and research usage prior to complete bio-material drying and final product preparation. However, since the dried remedies consist of near-perfectly preserved bio-compounds, the final commercial products and dried substances are highly recommended for any nutrient-, culinary-, herbal-, medical-, laboratory- and research purposes.

Our approach and methodology involves the utilization of collection containers filled with chemically inert gases and/or liquids cooled to temperatures from −30° C. to −180° C. that allow for instant and immediate cooling of any biological material collected and submersed within the enclosed volume of these containers—without addition of any antifreeze chemicals. A preferred choice of deep-freezing inert gases are nitrogen, carbon dioxide, neon, argon, xenon and a preferred choice of deep-freezing liquids are light-molecular weight hydrocarbons such as ethanol, methanol, ether, pentane, hexane, octane, decane, acetone etc. or mixes of the mentioned. While the liquefied nitrogen and solidified carbon dioxide are the least expensive, the hydrocarbon alcohols are more hydrophilic, they mix with the water and precipitate the biological content within the tissue on one hand while promoting the water evaporation during the drying phase—on the other hand. Overall, the liquid nitrogen remains the preferred choice because it offers the lowest possible freezing temperature at the range of −80° C. coupled with perfect chemical inactivity, which makes it an ideal carrier for biological substances and materials. However if extraction of aromatic oils, lipids phenols and other bio-substances is desired, the raw bio-material may preferably be submersed in deep-freezing alcohols or other organic solvents such as hydrocarbons.

The optimal setup for bio-material collection is its immediate preliminary processing at the growing field followed by submersion in the deep-freezing containers dedicated for preliminary storage employing submersion liquids (such as liquid nitrogen). The preliminary processing is performed preferably within 10-20 minutes after cutting-off the vital nutritions from the bio-matrial and includes but is not limited to separating and sorting desired parts and tissues (i.e. roots, stems, leafs, flowers, etc.), and/or reducing the material in size by cutting, chopping, mulching, shedding or else. In worse-case scenario, the collected biomaterial is cooled down to temperatures 5-20° C. for reducing the bio-degradation damage and transported to a closest processing facility where the preliminary processing is performed and the bio-material is immediately deep-frozen. The freezing at close to −180° C. (liquid nitrogen) leads to instant water glassification (vitrification) and near-perfect preservation of the entire biological content for as long as the liquid nitrogen is present in a liquid state within the thermostat container. For extended storage, dry carbon dioxide can be added to gradually replace the liquid nitrogen increasing the storage temperature to approximately −80° C. (that is still an excellent storage temperature to maintain) offering longer storage time since the solid carbon dioxide sublimates slower than the liquid nitrogen, it is easier to handle and cheaper to produce. Any addition of organic solvents must be done at temperatures near- or above the temperature of the dry carbon dioxide, since they will solidify immediately at so low temperatures (within the range of −176° C. to −79° C.).

Best technical setup for down-sizing the bio-material is by mulching and grinding it to a sand- or powder-size particles in metal grinders with double-wall chambers that allow a liquid nitrogen to be present between the double-walls for maintaining deep-frozen temperatures (at −170° C. range) while processing the bio-material.

The preparation of desired herbal remedies including combinations of different parts of plants, algae, lichens, fungi and/or molls, or combinations of entire species is performed immediately after the collection of the bio-material at the preliminary processing level (before the deep-freezing) and/or after the primary or secondary drying. Preferably, the preparation of combinatorial remedies is performed after the first-stage drying (at 5-10% of water content remaining within the material and low temperatures of 5-10° C.) since maintaining complete de-humidification is much more challenging and economically unreasonable at higher levels of bio-material drying (above 95% of water removal). However, the re-introduction of certain volatile compounds removed on earlier stages of cold bio-material processing (such as extracted aromatic oils, lipids, phenols, etc.) is preferably performed after the final drying of the bio-material and/or after the final composition of the commercial remedies—just before the encapsulation.

The preservation of all or any particular volatile (light-molecular-weight) bio-compound (such as, but not limited to aromatic light-molecular-weight oils, pheromones, lipids, phenols, etc.) are extracted directly from the biomaterial at a cold stage (−40 to −10° C.) with organic solvents (hydrocarbons) and/or extracted from the collected evaporated water after melting it at temperatures of 5° C. to 30° C. (but below the evaporation temperature of a particular light-weight bio-compound. The latest approach is employed for bio-compounds that remain together with the water vapor removed from the biological material due to the technical impossibility to be separated from the water vapor; these bio-substances are sub-sequentially removed from the water evaporate via all appropriate physical and chemical means, they are biophysically isolated and/or condensated, and re-introduced into the volume containing the dry biological material—where such an re-introduction is optional and/or desired for the final product preparation in cases of particular applications, but may not always be an obligatory option.

Further on, a first-stage drying of the grinded bio-material can be performed by transferring the frozen material into a vacuumization chamber or using the grinder's chamber as a vacuumization chamber where possible by design. As an optimal design, the vacuum-evaporation chamber must have double-walls maintaining the presence of liquid nitrogen or dry carbon dioxide for maintaining deep-freeze temperatures until more than 85% of the residual water is evaporated from the bio-material. The vacuum-dryer device must have a water condensation section to facilitate the removal of the evaporated water in order to maintain low humidity promoting the efficient drying of the bio-material. The vacuum dryer and the condenser might be electrically operated; however, water-absorbing materials may be implemented if desired upon appropriate design. Were convenient, the drying chamber may be equipped with a mixing device to promote equal surface access for all bio-material particles subjected to drying. Were reasonable—optical and electro-chemical sensors may be employed for monitoring the processes of bio-material disruption, drying and other manipulations. The initial step of drying is to achieve up to 85-95% of water sublimation and removal from the bio-material.

The secondary drying of the bio-material is employed for ensuring maximum preservation and increased shelf-life of the commercial products prepared. The secondary drying is performed after the removal of approximately 90% of the water-content from the bio-material. During the secondary drying the temperature within the drying chamber is gradually increased to promote efficient water removal. Depending upon the optimal technical design, the drying chamber for the secondary drying may have much large surface area for depositing the bio-material in order to promote the enhanced drying since it does not require to maintain deep-freezing temperatures. The presence of cold temperatures and vacuum are preferable but not obligatory and a gradual temperature increase from −50° C. up to +30° C. are desired. Increasing the temperature up to 0° C. are preferably performed by a gradual increase of the temperature with a rate of 5° C. per every 30-60 minutes if vacuum is applied to quickly remove the water vapor and with a rate of 5° C. per hour per each 1 kilogram of bio-material within the temperature range above 0° C. until reaching 15° C. Increasing the temperature above 15° C. up to as high as 40° C. must be performed in accordance with a humidity measurement, water-content of the sample and the specificity of the biological material processed. For perfect preservation of the bio-components the temperature of drying may not increase above 15° C.

For best preservation, all subsequent steps of transferring, sampling, mixing, stock-storage, encapsulation and vial-filling should preferably be performed at ambient temperature not above 15-18° C. and nitrogen- or other inert-gas atmosphere; ambient air is less desired due to its oxidation properties affecting many biological components. The dry biological material obtained by the described method is stored and processed under dry conditions into enclosed volumes (vessels, containers, bags, capsules etc.) ensuring a dry storage and/or (preferably) an absence of chemically harmful gases such as but not limited to oxygen, volatile chemical elements, molecules and vapors; thereby the storage volumes and conditions should maintain chemically inert atmosphere (preferably but not limited to vacuum- or nitrogen-), or any transitional atmospheric conditions between the last mentioned (i.e.—vacuum, nitrogen and/or other chemically inert gases or combinations of- and transitions between them) while handling the material of biological origin.

All bio-material manipulation stages require the implementation of design-appropriate equipment for volumes, cameras, chambers, ventilation, cooling, vapor entrapment and removal, absorption, extraction, lightening, sterilization, sensor and/or optical monitoring, and encapsulation and sampling.

### REFERENCES

1. Luyet, B. J. and Gehenio, P. M. (1940) Life and Death at Low Temperatures. Biodynamica, Normandy, MO.

2. Sutton, R. L. (1991) Critical cooling rates to avoid ice crystallization in solutions of cryoprotective agents. J. Chem. Soc. Faraday Trans. 87, 101-105.

3. Sutton, R. L. and Pegg, D. E. (1993) Devitrification in butane-2,3-diol solutions containing anti-freeze peptide. CryoLetters 14, 13-20.

4. Evans, S., Rachman, M. J., and Pegg, D. E. (1992) Design of a UHF applicator for rewarming of cryopreserved biomaterials. IEEE Transactions on Biomedical Engineering 39, 217-225.

5. Robinson, M. P., Wusteman, M. C., Wang, L. -H., and Pegg, D. E. (2002) Electromagnetic rewarming of cryopreserved tissues: effect of choice of cryoprotectant and sample shape on uniformity of heating. Physics in Medicine and Biology 47, 2311-2325.

6. Pegg, D. E., Wang, L. -H., and Vaughan, D. (2005) Cryopreservation of articular cartilage 2: Vitrification methods Cryobiology 51, Abstract 30.

7. Franks, F. (1981) Biophysics and biochemistry of low temperatures and freezing. In: Effects of Low Temperature of Biological Membranes, (Morris, G. J. and Clarke A., eds.), Academic Press, London, UK, pp. 3-19.

8. Smith, D. (1993) Tolerance to freezing and thawing. In: Stress Tolerance of Fungi, (Jennings, D. H., ed.), Marcel Dekker, Inc., New York, pp. 145-171.

9. Morris, G. J. (1981) Cryopreservation: An Introduction to Cryopreservation in Culture Collections. Culture Centre of Algae and Protozoa. ITE, Cambridge, UK.

10. Kirsop, B. E. and Doyle, A. (1991) Maintenance of Microorganisms and Cultured Cells: A Manual of Laboratory Methods. Academic Press, London, UK.

11. Smith, D., Ryan, M. J., and Day, J. G. (2001) The UKNCC Biological Resource: Properties, Maintenance, and Management. UKNCC Secretariat, Egham, Surrey, UK.

12. Hwang, S. -W. (1960) Effects of ultralow temperature on the viability of selected fungus strains. Mycologia 52,527-529.

13. Polge, C., Smith, A. U., and Parkes, S. (1949) Revival of spermatozoa after dehydration at low temperatures. Nature London 164,666.

14. Morris, G. J., Smith, D., and Coulson, G. E. (1988) A comparative study of the morphology of hyphae during freezing with the viability upon thawing of 20 species of fungi. J. Gen. Microbiol. 134, 2897-2906.

15. Smith, D. and Thomas, V. E. (1998) Cryogenic light microscopy and the development of cooling protocols for the cryopreservation of filamentous fungi. WJMB 14,49-57.

16. Hwang, S. -W. and Howell, A. (1968) Investigation of ultra-low temperature of fungal cultures II. Cryoprotection afforded by glycerol and dimethyl sulphoxide to eight selected fungal cultures. Mycologia 60,622-626.

17. Hwang, S. -W., Kylix, W. F., and Haynes, W. C. (1976) Investigation of ultra low temperature for fungal cultures III. Viability and growth rate of mycelial cultures cryogenic storage. Mycologia 68, 377-387.

18. Ashwood-Smith, M. J. and Warby, C. (1971) Studies on the molecular weight and cryoprotective properties of PVP and Dextran. Cryobiology 8,453-464.

19. Smith, D. (1983) Cryoprotectants and the cryopreservation of fungi. Trans. Br. Mycol. Soc. 80, 360-363.

20. Ryan, M. J. and Smith, D. (2004) Fungal Genetic Resource Centres and the genomic challenge. Mycol. Res. 108,1351-1362.

21. Pegg, D. E. (1987) Ice crystals in tissues and organs. In: The Biophysics of Organ Preservation, (Pegg, D. E. and Karow, A. M., Jr., eds.), Plenum Press, New York, pp. 117-140.

22. Taylor, M. J. and Pegg, D. E. (1983) The effect of ice formation on the function of smooth muscle tissue stored at −21° C. or −60° C. Cryobiology 20, 36-40.

23. Hunt, C. J., Taylor, M. J., and Pegg, D. E. (1982) Freeze-substitution and isothermal freeze-fixation studies to elucidate the pattern of ice formation in smooth muscle at 252 K (−21° C.). J. Micros. 125, 177-186.

24. Rubinsky, B. and Pegg, D. E. (1988) A mathematical model for the freezing process in biological tissue. Proc. R. Soc. Lond. 234,343-358.

25. Pegg, D. E., Wusteman, M. C., and Boylan, S. (1997) Fractures in cryopreserved elastic arteries.

26. Menges, M. and Murray, J. A. H. (2004) Cryopreservation of transformed and wild-type Arabidopsis and tobacco cell suspension cultures Plant J. 37, 635-644.

27. Withers, L. A. (1985) Cryopreservation of cultured plant cells and protoplasts. In: Cryopreservation of Plant Cells and Organs, (Kartha, K. K., ed.), CRC Press, Boca Raton, Fla., pp. 244-265.

28. Grout, B. (ed.) (1995) Genetic Preservation of Plant Cells in Vitro. Springer-Verlag, Berlin, Germany.

29. Fuller, B. J. (2004) Cryoprotectants: the essential antifreezes to protect life in the frozen state. CryoLetters 25, 375-388.

30. Mazur, P. (2004) Principles of cryobiology. In: Life in the Frozen State, (Fuller, B. J., Lane, N., and Benson, E. E., eds.), CRC Press, London, UK, pp. 3-66.

31. Muldrew, K., Acker, J. P., Elliott, J. A. W., and McGann, L. E. (2004) The water to ice transition: implications for living cells. In: Life in the Frozen State, (Fuller, B. J., Lane, N., and Benson, E. E., eds.), CRC Press, London, UK, pp. 67-108.

32. Withers, L. A. and King, P. J. (1980) A simple freezing unit and cryopreservation method for plant cell suspensions. CryoLetters 1, 213-220.

33. Danso, K. E. and Ford-Lloyd, B. V. (2004) Cryopreservation of embryogenic calli of cassava using sucrose cryoprotection and air desiccation. Plant Cell Rep. 22, 623-631.

34. Withers, L. A. (1990) Cryopreservation of plant cells. In: Methods in Molecular Biology Vol. 6: Plant Cell and Tissue Culture, (Pollard, J. W. and Walker, J. M., eds.), Humana Press, Totowa, N.J., pp. 39-48.

35. Benson, E. E. and Lynch, P. T. (1999) Cryopreservation of rice tissue cultures. In: Methods in Molecular Biology Vol. 111: Plant Cell Culture Protocols, (Hall, R. D., ed.), Humana Press, Totowa, N.J., pp. 383-394.

36. Shibli, R. A., Haagenson, D. M., Cunningham, S. M., Berg, W. K., and Volenec, J. J. (2001) Cryopreservation of alfalfa (Medicago sativa L.) by encapsulation-dehydration. Plant Cell Rep. 20, 445-450.

37. Lambardi, M., Lynch, P. T., Benelli, C., Mehra, A., and Siddika, A. (2002) Towards the cryopreservation of olive germplasm. Advances in Hort. Science 16, 165-174.

38. Schrijnemakers, E. W. M. and Van Iren, F. (1995) A two-step or equilibrium freezing procedure for the cryopreservation of plant cell suspensions. In: Methods in Molecular Biology vol. 38, Cryopreservation and Freeze-drying Protocols, (Day, J. G. and McLellan, M. R., eds.), Humana Press, Totowa, N.J., pp. 103-111.

39. Sakai, A. (2004) Plant cryopreservation. In: Life in the Frozen State, (Fuller, B. J., Lane, N., and Benson, E. E., eds.), CRC Press, London, UK, pp. 329-346.

40. Leunufna, S. and Keller, E. R. S. (2005) Cryopreservation of yams using Vitrification modified by including droplet method: effects of cold acclimation and sucrose. CryoLetters 26, 93-102.

41. Wang, Q. Mawassi, M., Sahar, N., et al. (2004) Cryopreservation of grapevine (Vitis spp.) embryogenic cell suspensions by encapsulation-vitrification. Plant Cell Tissue Organ Cult. 77, 267-275.

42. Mazur, P. (2004) Principles of cryobiology. In: Life in the Frozen State, (Fuller, B., Lane, N., and Benson, E. E., eds.), CRC Press, Boca Raton, Fla., pp. 3-66.

43. Benson, E. E., Johnston, J., Muthusamy, J., and Harding, K. (2005) Physical and engineering perspectives of in vitro plant cryopreservation. In: Plant Tissue Culture Engineering, (Dutta Gupta, S. and Ibaraki, Y., eds.), Springer, Netherlands, pp. 441-476.

44. Benson, E. E. (1999) Plant Conservation Biotechnology. Taylor and Francis, London, UK.

45. Benson, E. E. (2004) Cryo-conserving plants and algae: historical perspectives and future challenges. In: Life in the Frozen State, (Fuller, B., Lane, N. and Benson, E. E., eds.), CRC Press, Boca Raton, Fla., pp. 299-328.

46. Fuller, B., Lane, N., and Benson, E. E. (2004) Life in the Frozen State. CRC Press, Boca Raton, Fla.

What is claimed is the following:

1. A method for obtaining biological material and substances followed by commercially viable preparation and preservation of plants, funguses, lichens and algae, entire plant-, fungus-, lichen- or algae material, parts of entire plant-, fungus-, lichen- or algae species and/or any desired combinations of these species or parts of them, or mixes of them including but not limited to those serving herbal, medicinal and supplemental properties and purposes, with sad method comprising of the following steps, manipulations and conditions listed below in a non-successive order:
rapid instantaneous deep freezing of the entire plant-, fungus-, lichen- and/or algae material used for commercial purposes shortly after the material collection, within minutes (less than two hours, but preferably within 20 minutes or less), before the vitality of the cells is destroyed,—by the means of using (preferably but not limited to) liquefied and/or solidified deep-freezing liquids or gases to momentarily submerse the biological material with their deep-cold solid-, liquid- or gaseous state (molecules) in certain enclosed volumes resulting in instant rapid deep freezing of the biological material to temperatures below minus−30 degree Celsius (° C.), and by the means of using preferably but not limited to liquid nitrogen (at minus−180° C. to minus−170° C.), dry ice (carbon dioxide) (at minus−80° C. to minus−30° C.) or else (below minus−30° C.)—thereby preserving the prevailing majority of both the cell structure and content as in the state of vitality mainly as a result of so called water “glassification” (vitrification) process (i.e. water freezing without crystallization, volume increase, and morphological and chemical structure disruption of the biomaterial);
storing, transporting, grinding or else treating the sad plant-, fungus-, lichen- and/or algae material and/or mixing different species materials or parts of them under conditions preferably (but not limited to) being submerged in liquid nitrogen, dry ice or otherwise at temperatures not increasing above the minus−20° C. until the containing water is completely evaporated from the biological material by sublimation (from solid—to gaseous form) and removed from the volume (vessel) containing the dry biological material;
drying the frozen (below the minus−20° C.) biological material entirely by sublimation of the containing water molecules from the biological material into the surrounding air followed by further removal of the water vapor out of the bio-material-containing volume (vessel) by means including but not limited to removal by vacuumization, adsorption into hydroscopic substrates (materials), ventilation and liquefaction (condensation) on acceptor surfaces & materials, etc. and/or combination of the above;
mechanical processing of the biological material in enclosed volumes under completely dry condition maintained by vacuum, chemically inert gases such as but not limited to nitrogen and/or else atmosphere—such a processing including but not limited to chopping, grinding, weighting, transferring, storage-vessel filling, capsule filling, tablet making, encapsulation within hard-surface materials capable of maintaining further dry conditions, preventing contacts with volatile solutions, gases and/or else under cold- or ambient temperatures not increasing above 60° C.

2. A method of claim 1 comprising a step of rapid instantaneous deep freezing of the entire plant-, fungus-, lichen- and/or algae material used for commercial purposes shortly after the material collection, within minutes (less than two hours, but preferably within 20 minutes or less), before the vitality of the cells is destroyed,—by the means of submersing the bio-material using frozen hydrocarbons and their mixes with or without liquefied and/or solidified gases or else (such as but not limited to chemically inert deep-frozen liquids or gases, instant and direct contact to deep-frozen molecules, chunks and/or surfaces, etc.), and/or by direct and instant contact of the biological material to pre-cooled surfaces providing instant cooling of the biological material to temperatures below minus−30 degree Celsius (° C.)—thereby preserving the prevailing majority of both the cell structure and content as in the state of vitality mainly as a result of so called water “glassification” (vitrification) process (i.e. water freezing without crystallization, volume increase, and morphological and chemical structure disruption of the biomaterial).

3. A method described in claim 1 combined or not with additional procedures of collecting (in instance or in selective order) all the co-evaporated and co-separated biological substances that originate from the processed plant-, fungus-, lichen- and/or algae material (including but not limited to aromatic and/or light-molecular-weight oils, pheromones, etc.) and that remain together with the water vapor removed from the biological material due to the technical impossibility to be separated from the water vapor; by the sub-sequential removal of these bio-substances from the water evaporate via all appropriate physical and chemical means, their biophysical isolation and/or condensation, and re-introduction into the volume containing the dry biological material—where such an re-introduction is optional and/or desired for the final product preparation in cases of particular applications but may not always be obligatory option.

4. Storing the biological material obtained by the method described in claim 1 under dry conditions into enclosed volumes (vessels, containers, bags, capsules etc.) ensuring a dry storage and/or (preferably) an absence of chemically harmful gases such as but not limited to oxygen, volatile chemical elements, molecules and vapors; thereby the storage volumes and conditions should maintain chemically inert (preferably but not limited to vacuum- or nitrogen-) atmosphere, or any transitional conditions between the last mentioned (vacuum, nitrogen and/or other chemically inert gases or combinations of—and transitions between them) while handling the material of biological origin.

5. A method described in claim 1 for commercial preparation and preservation of plants, funguses, lichens and/or algae, entire plant-, fungus-, lichen- and/or algae material, parts of the plant-, fungus-, lichen- and/or algae species and/or any desired combinations of different biological species or parts of them, including but not limited to those serving herbal, medicinal and supplemental properties and purposes, with sad method serving for the preparation of commercially viable substances and mixes of plant- and/or fungal origin rather than intended for laboratory and/or research usage.

6. A method described in claim 1 for commercial preparation and preservation of plants, funguses, lichens and/or algae, entire plant-, fungus-, lichen- and/or algae material, parts of the plant-, fungus-, lichen- and/or algae species and/or any desired combinations of different biological species or parts of them with such a method serving biochemical and biophysical conditions for total and complete, biological and chemical conservation and preservation of the entire plant- and/or fungal content—practically identical to those existing in the nature before the vitality state is halted and/or destroyed—and within the prevailing majority of the biological material allowing for maintaining the vitality-potential conditions of the cells, cell structures and cellular content (within the mechanically processed biological material) that are finally prepared for commercial usage and/or market introduction—without a special introduction of antifreeze compounds for the purpose of vitality stabilization on any stage of the biomaterial processing.

7. A method described in claim 1 for commercial preparation and preservation of plants, funguses, lichens and/or algae, entire plant-, fungus-, lichen- and/or algae material, parts of entire plant-, fungus-, lichen- and/or algae species and/or any desired combinations of different biological species or parts of them, which method allows for post-conservational (after-freezing) sub-sequential manipulation of the biological (plant-, fungus-, lichen- and/or algae-) material for the extraction and/or separation, or other manipulation of the biological material with the intention but not limited to isolation, purification or else usage and utilization of any particular or combinative biological component(s) of the biological material intended for commercial, medicinal and/or supplemental purposes rather than for any laboratory research investigations prior to the final dried-product preparation.

8. The commercial product manipulation according to claim 1 which is frozen and sheared simultaneously and/or successively in a vessel comprising a rotor with attached shear blades with the rotor attached to a motor wherein said rotor comprises an impeller of correct proportions to scrape the surface near the edge of the vessel as the impeller rotates under ambient or frozen conditions thereby successfully removing any bio-material deposits from the surface of the vessel—in the presence or absence of inert atmospheric conditions.

9. The commercial product according to claim 1 is frozen and sheared in a device vessel (chamber) equipped with rotating blade(s) and double walls designed to maintain the presence and re-filling of a deep-freeze coolant such as liquid nitrogen, dry carbon dioxide, liquid hydrocarbons and/or silicones—inside the shredding chamber or between the double walls of the vessel chamber.

10. A method for commercial preparation and preservation of plants, funguses, lichens and/or algae, entire plant-, fungus-, lichen- and/or algae material, parts of entire plant-, fungus-, lichen- and/or algae species and/or any desired combinations of different biological species or parts of them, including but not limited to those serving herbal, medicinal and/or supplemental properties and purposes, during the sad method the entire process of processing the plant and/or fungal material from it's obtaining to it's inclusion (packing) in market-oriented vessels (encapsulation) and commercial shipment maintaining the following conditions: temperatures below minus−20° C. (preferably, between minus−180 to minus−40 degree Celsius) if residual water is present within the plant material or temperatures below +60° C. (preferably, within a temperature maximum of +15 to +30° C.)—in the absence of water (dry state), and preferably maintaining vacuum or chemically inert atmosphere of gases and/or else vapor (preferable but not limited to nitrogen) for minimizing the oxidation and/or other chemical alteration.
